(19) United States
(12) Patent Application Publication
(10) Pub. No.: US 2012/0309684 A1
(43) Pub. Date: Dec. 6, 2012

(54) CONJUGATES FOR DELIVERY OF BIOLOGICALLY ACTIVE COMPOUNDS

(75) Inventors: Matthew Wood, Oxford (GB); Haifang Yin, Oxford (GB)

(73) Assignee: ISIS INNOVATION LIMITED, OXFORD (GB)

(21) Appl. No.: 13/512,432

(22) PCT Filed: Nov. 26, 2010
(86) PCT No.: PCT/GB10/02187

§ 371 Date: Aug. 9, 2012

Related U.S. Application Data
(60) Provisional application No. 61/264,960, filed on Nov. 30, 2009.

(51) Int. Cl. A61K 38/02; C12N 15/85; A61P 21/00; C07K 9/00; C07K 19/00; A61P 9/00; C12N 5/071; C07K 2/00

(52) U.S. Cl. 514/16.4; 435/375; 435/320.1; 530/300; 530/322; 530/350; 514/1.1

(57) ABSTRACT

A construct suitable for delivery of a biologically active compound into cells, comprising:
(d) a positively charged peptide;
(e) a targeting-delivery peptide; and
(f) the biologically active compound;
   wherein the positively charged peptide is covalently attached to the targeting-delivery peptide and the biologically active compound is covalently or non-covalently attached to the resultant chimeric cell delivery peptide.

### FIELD OF THE INVENTION

The present invention relates to delivering molecules into a cell.

### BACKGROUND OF THE INVENTION

There is a need in the art for improved methods of facilitating uptake of compounds into cells, particularly to deliver therapeutic compounds to cells.

### SUMMARY OF THE INVENTION

The invention is based on characterisation of properties of substances that could facilitate delivery of compounds into cells.

The inventors have shown that chimeric cell delivery peptides comprising a positively charged peptide and a targeting-delivery peptide are capable of highly efficient delivery of biologically active compounds into cells. Accordingly the invention provides a construct suitable for delivery of a biologically active compound into cells, comprising:
(a) a positively charged peptide;
(b) a targeting-delivery peptide; and
(c) the biologically active compound;
   wherein the positively charged peptide is covalently attached to the targeting-delivery peptide and the biologically active compound is covalently or non-covalently attached to the resultant chimeric cell delivery peptide.

### BRIEF DESCRIPTION OF THE FIGURES

FIG. 1 shows the systemic administration of MSP-PMO and B-PMO conjugates in mdx mice. Dystrophin expression following single 25 mg/kg intravenous injections of the B-PMO and MSP-PMO AO conjugates in adult mdx mice. (a) Schematic figure illustrating the 4 different AO constructs utilised. PMO contains the sequence of GGCCAAACCTCGGCTTACCTGAAAT (5′-3′; SEQ ID NO: 54). Peptides are written from N to C orientation using the standard one letter amino acid code except for X and B, which are un-natural amino acids (X=6-aminohexanoic acid, B=beta-alanine). (b) Immunostaining of muscle tissue cross-sections to detect dystrophin protein expression and localisation in C57BL6 normal control (top panel), untreated mdx mice (middle panel), B-PMO treated (third panel) and MSP-PMO treated mdx mice (bottom panel). Muscle tissues analysed were from tibialis anterior (TA), gastrocnemius, quadriceps, biceps, abdominal wall (abdominal), diaphragm and heart muscles (scale bar=200 μm).

FIG. 2 shows an investigation of muscle-specific chimeric peptide PMO conjugates at low systemic doses. Dystrophin exon-skipping and protein expression following systemic administration of muscle-specific fusion peptide PMO conjugates in adult mdx mice. (a) Immunohistochemistry to detect dystrophin expression in muscle cross-sections from mdx mice treated with B-PMO (upper panel), B-MSP-PMO (second panel) and MSP-B-PMO (lower panel) conjugates at the low 3 mg/kg dose. Data from control normal C57BL6 and untreated mdx mice not shown. Muscle tissues analysed were from tibialis anterior (TA), gastrocnemius, quadriceps, biceps, abdominal wall (abdominal), diaphragm and heart muscles (scale bar=200 μm). Dystrophin expression was not found in heart with all 3 conjugates at this dose. (b) RT-PCR to detect the dystrophin exon skipping products in treated mdx mouse muscle groups as shown (exon-skipped bands indicated by Δexon23—for exon 23 deleted; Δexon22+23—for exons 22 and 23 deleted). (c) Sequence analysis confirming precise skipping of exon 23 and another RT-PCR product with both exon 22 and 23 skipped. (d) Western blot for detection of dystrophin protein in the indicated muscle groups from treated mdx mice compared with C57BL6 and untreated mdx control mice. 100 μg protein was loaded for each sample except for C57BL6 control lane where 1 μg of protein was loaded. α-actinin was used as loading control.

FIG. 3 shows that systemic administration of the B-MSP-PMO conjugate restores dystrophin expression in body-wide skeletal muscles. Dystrophin exon-skipping and protein expression following systemic administration of the B-MSP-PMO conjugate in adult mdx mice at a dose of 6 mg/kg. (a) Immunohistochemistry to detect dystrophin expression in muscle cross-sections from mdx mice treated with B-PMO (top panel), B-MSP-PMO (bottom panel) conjugates at the 6 mg/kg dose. Data from control normal C57BL6 and untreated mdx mice are not shown. Muscle tissues analysed were from tibialis anterior (TA), gastrocnemius, quadriceps, biceps, abdominal wall (abdominal), diaphragm and heart muscles (scale bar=200 μm). Widespread, uniform dystrophin expression detected in all skeletal muscles treated with the B-MSP-PMO conjugate, however low level of dystrophin expression was found in heart. (b) RT-PCR to detect dystrophin exon skipping products in treated mdx muscle groups as shown (Δexon23 indicates exon 23 deleted; Δexon22+23—exons 22 and 23 deleted). (c) Western blot detection of dystrophin protein in the indicated muscle groups from treated mdx mice compared with C57BL6 and untreated mdx control. Equal loading of 25 μg protein is shown for each sample except for C57BL6 control lane where 6.25 μg of protein was loaded and α-actinin as a loading control. (d) Quantification of dystrophin protein levels relative to normal controls in differently treated muscles. The mean percentage of dystrophin protein relative to normal control restored in different muscles treated with B-MSP-PMO was 24.3%, 20.1%, 15.7%, 19.3%, 17.2%, 1.7% and 14.5% in TA, quadriceps, gastrocnemius, biceps, diaphragm, heart and abdominal muscle respectively, in comparison with the 9.9%, 6.9%, 4.2%, 5.9%, 4%, 2.5% and 6.9% in the B-PMO treated mice (the percentage is shown as mean+SEM, n=4 mice). is a western blot analysis. Total protein was extracted from TA muscles of 2-month old mdx mice two weeks after a single intramuscular injection with 5 μg PNA-peptide conjugate. No visible difference in the size of dystrophins between muscle treated with PNA and muscle from the normal C57BL6 mouse.

FIG. 4 shows the functional and phenotypic correction in mdx mice following treatment with the B-MSP-PMO conjugate. (a) Restoration of the dystrophin-associated protein complex (DAPC) in mdx mice treated with B-MSP-PMO at 6 mg/kg was studied to assess dystrophin function and recovery of normal myoarchitecture. DAPC protein components β-dystroglycan, α and β-sarcoglycan and nNOS were detected by immunostaining in serial tissue cross-sections of TA muscles from treated mdx mice compared with B-PMO treated mdx mice (arrowhead indicated identical fibres). (b) Muscle function was assessed using a functional grip strength test to determine the physical improvement of B-MSP-PMO treated mdx mice compared with untreated controls and B-PMO treated mdx mice showing close correlation with the percentage of dystrophin-positive fibres in biceps muscles (R²=0.8007). (c) Evaluation of the numbers of centrally nucleated myofibres in TA, gastrocnemius and quadriceps muscles following B-MSP-PMO treatment compared with the corresponding untreated mdx muscles. Data shows a significant decrease in the number of centrally nucleated myofibres in treated mdx muscles compared with untreated controls (P<0.001). (d) Measurement of serum creatine kinase (CK) levels as an index of ongoing muscle membrane instability in treated mdx mice compared with mdx control mice. Data shows a significant fall in the serum CK levels in mdx mice treated with B-MSP-PMO compared with untreated age-matched mdx controls (P<0.05). (e) Measurement of serum levels of aspartate aminotransferase (AST) and alanine aminotransferase (ALT) enzymes in treated mdx mice compared with untreated mdx mice. Data shows improved pathological parameters in B-MSPPMO treated mdx mice compared with untreated controls with significantly lower serum levels of both enzymes.

FIG. 5 shows systemic administration of 9-B-PMO and B-9-PMO conjugates in adult mdx mice. Dystrophin expression following single 25 mg/kg intravenous injections of the 9-B-PMO and B-9-PMO conjugates in young adult mdx mice. (a) Immunostaining of muscle tissue cross-sections to detect dystrophin protein expression and localisation in C57BL6 normal control (top panel), untreated mdx mice (second panel), 9-B-PMO treated (third panel) and B-9-PMO treated mdx mice (bottom panel). Muscle tissues analysed were from tibialis anterior (TA), gastrocnemius, quadriceps, biceps, diaphragm, heart and abdominal wall (abdominal) muscles (scale bar=200 μm). (b) Quantification of dystrophin-positive fibres in muscle cross-sections from mdx mice treated with 25 mg/kg 9-B-PMO and B-9-PMO. The data is presented as mean±SEM and significant difference was observed in B-9-PMO treated mdx mice compared with 9-B-PMO (t-test, *P<0.05; n=4). (c) RT-PCR to detect exon skipping efficiency at the RNA level. Exon skipping products are shown by shorter exon-skipped bands (indicated by Δexon23—exon 23 deleted; Δexon22&23—both exon 22 and 23 skipped). (d) Western blot for dystrophin expression in 9-B-PMO and B-9-PMO treated mdx mice. Equal loading of 10 μg protein is shown for each sample except for the C57BL6 control lanes where 5 and 2.5 μg protein was loaded, respectively. α-actinin was used as loading control.

FIG. 6 shows systemic administration of Pip5e-MSP-PMO conjugates in adult mice. (a) Immunostaining of muscle tissue cross-sections to detect dystrophin protein expression and localisation. The graph shows quantification of dystrophin-positive fibres in muscle cross-sections in each muscle tissue. Muscle tissues analysed were from tibialis anterior (TA), gastrocnemius, quadriceps, biceps, diaphragm, heart and abdominal wall (abdominal) muscles. (b) RT-PCR to detect exon skipping efficiency at the RNA level (top gel). Western blot for dystrophin expression (bottom gels).

FIG. 7 shows systemic administration of RXB-MSP-RXB-PMO conjugates in adult mice. (a) Immunostaining of muscle tissue cross-sections to detect dystrophin protein expression and localisation. The graph shows quantification of dystrophin-positive fibres in muscle cross-sections in each muscle tissue. Muscle tissues analysed were from tibialis anterior (TA), gastrocnemius, quadriceps, biceps, diaphragm, heart and abdominal wall (abdominal) muscles. (b) RT-PCR to detect exon skipping efficiency at the RNA level (top gel). Western blot for dystrophin expression (bottom gels).

### DESCRIPTION OF SEQUENCES

The sequence of the human dystrophin gene and the location of the exons and introns can be obtained from the following web link: http://vega.sanger.ac.uk/Homo_sapiens/transview?transcript=OTTHUMT00000056182

The partial sequence of the mouse dystrophin gene and the full intron sequences can be accessed at the following web link: http://vega.sanger.ac.uk/Mus_musculus/transview?transcript=OTTMUST00000043357

SEQ ID NOs: 1 to 44 show preferred sequences for inclusion in the positively charged peptide.

SEQ ID NOs: 45 to 49 show preferred sequences for inclusion in the targeting-delivery peptide.

SEQ ID NOs: 50 to 53 show preferred chimeric sequences for used in the invention.

SEQ ID NO: 54 shows the sequence of PMO used in the Examples.

SEQ ID NO: 55 to 177 are exon/intron boundary sequences that can be targeted by antisense oligonucleotide sequences.

### DETAILED DESCRIPTION OF THE INVENTION

Peptide-mediated cell delivery is the use of a peptide or peptides, either as non-covalent complexes or as covalent conjugates, to enhance the delivery of molecules, such as a biologically active compound, into cells. A peptide capable of effecting peptide-mediated cell delivery may be referred to as a “cell delivery peptide” or a “cell penetrating peptide”. Examples of cell delivery peptides may include tissue-specific peptides (such as MSP) or transduction peptides (such as HIV TAT peptide).

The inventors have discovered novel constructs suitable for delivery of a biologically active compound into cells, such as cardiac and skeletal muscle cells. The cell delivery peptide constructs comprise a positively charged peptide linked to a targeting-delivery peptide. The chimeric cell delivery peptide is linked to a biologically active compound. The presence of the positively charged peptide increases the efficiency of delivery of the biological compound by the targeting-delivery peptide.

These constructs can be used to deliver the biologically active compound into a cell in vivo or in vitro, and may be used in a method of treatment or diagnosis of the human or animal body. In particular, the constructs deliver a biologically active compound to cardiac and heart muscle cells, and therefore the constructs may be used in a method of treatment or diagnosis of a cardiac or skeletal muscle disease.

### Positively Charged Peptide

The positively charged peptide may be any peptide that has a net positive charge. In one embodiment, the positively charged peptide is a straight (i.e. unbranched) chain of amino acids. The straight chain is typically from 6 to 30 amino acids, such as from 8 to 25 amino acids or from 10 to 20 amino acids, in length.

The positively charged peptide is typically rich in positively charged amino acids. A positively charged amino acid is an amino acid with a net positive charge. The positively charged amino acids can be naturally occurring or non-naturally occurring. The positively charged amino acids may be synthetic or modified. For instance, modified amino acids with a net positive charge may be specifically designed for use in the invention. A number of different types of modification to amino acids are well known in the art. Preferred naturally occurring positively charged amino acids include, but are not limited to, histidine (H), lysine (K) and arginine (R). Any number and combination of H, K and/or R may be present in the positively charged peptide.

A positively charged peptide is “rich” in positively charged amino acids if at least 20%, such as at least 30%, at least 40%, at least 50%, at least 60%, at least 70% or at least 80%, of its amino acids are positively charged. In a preferred embodiment, at least 20% of the amino acids in the positively charged peptide are arginine (R).

The positively charged peptide preferably comprises a sequence of the formula (RZR(Z)ₗ(ILFQY)ₘ)ₙ or a functional derivative thereof, wherein Z is an aminoalkyl spacer, l is 0 or 1, m is 0 or 1 and n is from 2 to 6. I=isoleucine, L=leucine, F=phenylalanine, Q=glutamine, Y=tyrosine.

An aminoalkyl spacer is a molecule that can separate amino acids in the peptide chain. The aminoalkyl spacer may have from 1 to 6, such as 2, 3, 4 or 5, carbon atoms. The aminoalkyl spacer typically comprises an amino group and a carboxyl group such that it can bind to the adjacent amino acids in the peptide chain though peptide bonds. Preferred aminoalkyl spacers include, but are not limited to, 6-aminohexanoyl (X), betaalanyl (B), 4-aminobutyryl, p-aminobenzoyl, or isonipecotyl.

The positively charged peptide preferably comprises two or more RZR groups (for example RXR and/or RBR groups). The number of these groups is determined by the value of n. n is from 2 to 6, such as 3, 4 or 5. n is preferably 3. For each value of n in a positively charged peptide, the Z in RZR may independently be X or B. For instance, the positively charged peptide may comprise the sequence RXRRXR (SEQ ID NO: 1), RBRRBR (SEQ ID NO: 2), RXRRBR (SEQ ID NO: 3) or RBRRXR (SEQ ID NO: 4).

The two or more RXR and/or RBR groups may be separated by Z (if l is 1) and/or ILFQY (if m is 1). For each value of n, if l is 1, m is preferably 0. For each value of n, if m is 1, l is preferably 0. For each value of n in a positively charged peptide, the separating group may independently be Z or ILFQY. For instance, if n is 3, the peptide may comprise the sequence RXRZRXRILFQYRXR (i.e where the first two RZRs are separated by Z and the second two RZRs are separated by ILFQY; RXRZ-SEQ ID NO: 5).

If n is 2 or 3, the positively charge peptide may comprise one or more of the sequences shown in Table 1.

TABLE 1
Preferred positively charged sequences

| SEQ ID NO: | Sequence |
|---|---|
| 1 | RXRRXR |
| 2 | RBRRBR |
| 3 | RXRRBR |
| 4 | RBRRXR |
| — | RXRZRXR |
| — | RBRZRBR |
| — | RXRZRBR |
| — | RBRZRXR |
| 5 | RXRILFQYRXR |
| 6 | RBRILFQYRBR |
| 7 | RXRILFQYRBR |
| 8 | RBRILFQYRXR |
| 9 | RXRRXRRXR |
| 10 | RBRRBRRBR |
| 11 | RXRRBRRXR |
| 12 | RXRRBRRBR |
| 13 | RXRRXRRBR |
| 14 | RBRRXRRBR |
| 15 | RBRRXRRXR |
| 16 | RBRRBRRXR |
| RXRZ-SEQ ID NO: 1 | RXRZRXRRXR |
| SEQ ID NO: 1-ZRXR | RXRRXRZRXR |
| 17 | RXRILFQYRXRRXR |
| 18 | RXRRXRILFQYRXR |
| — | RXRZRXRZRXR |
| 19 | RXRILFQYRXRILFQYRXR |
| SEQ ID NO: 5-ZRXR | RXRILFQYRXRZRXR |
| RXRZ-SEQ ID NO: 5 | RXRZRXRILFQYRXR |
| RBRZ-SEQ ID NO: 2 | RBRZRBRRBR |
| SEQ ID NO: 2-ZRBR | RBRRBRZRBR |
| 20 | RBRILFQYRBRRBR |
| 21 | RBRRBRILFQYRBR |
| SEQ ID NO: 22-ZRBR | RBRYRBRZRBR |
| 23 | RBRILFQYRBRILFQYRBR |
| RBRZ-SEQ ID NO: 6 | RBRZRBRILFQYRBR |
| SEQ ID NO: 6-ZRBR | RBRILFQYRBRZRBR |
| RXRZ-SEQ ID NO: 4 | RXRZRBRRXR |
| SEQ ID NO: 4-ZRXR | RXRRBRZRXR |
| 24 | RXRILFQYRBRRXR |
| 25 | RXRRBRILFQYRXR |
| — | RXRZRBRZRXR |
| 26 | RXRILFQYRBRILFQYRXR |
| RXRZ-SEQ ID NO: 8 | RXRZRBRILFQYRXR |
| SEQ ID NO: 7-ZRXR | RXRILFQYRBRZRXR |
| RXRZ-SEQ ID NO: 2 | RXRZRBRRBR |
| SEQ ID NO: 3-ZRBR | RXRRBRZRBR |
| 27 | RXRILFQYRBRRBR |
| 28 | RXRRBRILFQYRBR |
| — | RXRZRBRZRBR |
| 29 | RXRILFQYRBRILFQYRBR |
| RXRZ-SEQ ID NO: 6 | RXRZRBRILFQYRBR |
| SEQ ID NO: 7-ZRBR | RXRILFQYRBRZRBR |
| RXRZ-SEQ ID NO: 3 | RXRZRXRRBR |
| SEQ ID NO: 1-ZRBR | RXRRXRZRBR |
| 30 | RXRILFQYRXRRBR |
| 31 | RXRRXRILFQYRBR |
| — | RXRZRXRZRBR |
| 32 | RXRILFQYRXRILFQYRBR |
| RXRZ-SEQ ID NO: 7 | RXRZRXRILFQYRBR |
| SEQ ID NO: 5-ZRBR | RXRILFQYRXRZRBR |
| RBRZ-SEQ ID NO: 3 | RBRZRXRRBR |
| SEQ ID NO: 4-ZRBR | RBRRXRZRBR |
| 33 | RBRILFQYRXRRBR |
| 34 | RBRRXRILFQYRBR |
| — | RBRZRXRZRBR |
| 35 | RBRILFQYRXRILFQYRBR |
| RBRZ-SEQ ID NO: 7 | RBRZRXRILFQYRBR |
| SEQ ID NO: 8-ZRBR | RBRILFQYRXRZRBR |
| RBRZ-SEQ ID NO: 1 | RBRZRXRRXR |
| SEQ ID NO: 4-ZRXR | RBRRXRZRXR |
| 36 | RBRILFQYRXRRXR |
| 37 | RBRRXRILFQYRXR |
| — | RBRZRXRZRXR |
| 38 | RBRILFQYRXRILFQYRXR |
| RBRZ-SEQ ID NO: 5 | RBRZRXRILFQYRXR |
| SEQ ID NO: 8-ZRXR | RBRILFQYRXRZRXR |
| RBRZ-SEQ ID NO: 4 | RBRZRBRRXR |
| SEQ ID NO: 2-ZRXR | RBRRBRZRXR |
| 39 | RBRILFQYRBRRXR |
| 40 | RBRRBRILFQYRXR |
| — | RBRZRBRZRXR |
| 41 | RBRILFQYRBRILFQYRXR |
| RBRZ-SEQ ID NO: 8 | RBRZRBRILFQYRXR |
| SEQ ID NO: 6-ZRXR | RBRILFQYRBRZRXR |

Based on the sequence of the specific peptides shown in Table 1, a person skilled in the art can easily envisage peptides for use in the invention where n is 4, 5 and 6.

The positively charge peptide preferably comprises one or more of the sequences shown in Table 2.

TABLE 2
Other preferred positively charged sequences

| SEQ ID NO: | Sequence |
|---|---|
| 42 | RXRRXRRXRRXR |
| 43 | RXRRBRRXRILFQYRXRBRXRB |
| 44 | RXRRBRRXRRBRXB |

The positively charge peptide may be one of the peptide nucleic acid (PNA) or phosphorodiamidate morpholino oligonucleotide (PMO) internalization peptides (PIPs) known in the art. Suitable peptides are disclosed in Ivanova et al., Nucleic Acids Research, 2008; 36(20): 6418-6428. In a preferred embodiment, the positively charged peptide comprises the sequence of PIP5. In a more preferred embodiment, the positively charged peptide comprises the sequence of PIP5e (SEQ ID NO: 43).

In another embodiment, the positively charged peptide is a branched peptide. The branched peptide may comprise two or more, such as 3 or 4, chains of peptide. The chains of peptide may be the same or different. Each chain of peptide may comprise any of those sequences discussed above. For instance, a branched peptide may comprise two chains comprising the sequence shown in SEQ ID NO: 11 or may comprise a first chain comprising the sequence shown in SEQ ID NO: 6 and a second chain comprising the sequence shown in SEQ ID NO: 7.

Branched peptides may be formed using any method known in the art. In a preferred embodiment, a lysine (K) residue is used to branch two peptide chains. One chain is attached to the alpha amino position of the K residue and the other chain is attached to the epsilon position of the K residue. In another preferred embodiment, three lysine (K) residues are used to branch four chains. One K residue is used as the base. One K residue is attached to the alpha amino position of the base K residue and the third K residue is attached to the epsilon position of the base K residue. Peptide chains can then be attached to the each of the four amino positions of the two K residues “linked” by the base K residue.

### Targeting-Delivery Peptide

The targeting-delivery peptide is preferably selected from MSP, HSP, AAV6, AAV8 and TAT or a functional derivative thereof.

Muscle-specific protein (MSP) is a 7mer muscle-specific peptide, originally identified by screening a phage library in the mouse cell line C2C12, and here evaluated as a potential delivery peptide for the first time. The MSP peptide is ASSLNIA (SEQ ID NO: 45).

The HSP peptide is SKTFNTHPQSTP (SEQ ID NO: 46).

AAV6 is a 21mer peptide derived from a putative heparin-binding domain on the surface loop of the AAV6 capsid protein VP1 (576-597). AAV6 is reported to transfect skeletal muscle with high efficiency but its detailed structure is still unavailable. The AAV6 capsid protein VP1 was therefore compared with the well-characterised AAV2 capsid protein VP1 which identified the putative heparin-binding domain for cell tropism by bioinformatic analysis of AAV serotypes 1, 2, 6, 7 and 8 (data not shown). Another 21mer peptide (578-599) from the AAV8 capsid protein VP1 was also identified through the same bioinformatic analysis. AAV8 has been reported to be highly effective at transfecting skeletal and cardiac muscle. The AAV6 peptide is TVAVNLQSSSTDPATGDVHVM (SEQ ID NO: 47). The AAV8 peptide is IVADNLQQQNTAPQIGTVNSQ (SEQ ID NO: 48).

The TAT peptide is YGRKKRRQRRRP (SEQ ID NO: 49). HIV TAT (referred to as TAT) is a well-studied 12mer peptide that has been previously tested for delivering a range of different oligonucleotides in vitro and in vivo.

### Attachment

The positively charged peptide is covalently attached to the targeting-delivery peptide to form a peptide chimera. This can be done using any method in the art. The positively charged peptide may be covalently attached to the amino terminus or the carboxy terminus of the targeting-delivery peptide. The positively charged peptide is preferably covalently attached to the amino terminus of the targeting-delivery peptide.

The peptides can be covalently attached using a linker. Suitable linkers are well known in the art. Suitable linkers include, but are not limited to, chemical crosslinkers and peptide linkers. The peptides are preferably linked by two or more, such as 3, 4, 5 or 6 amino acids.

In one preferred embodiment, the positively charged peptide is genetically fused to the targeting-delivery peptide. The peptides are genetically fused if the peptide chimera (i.e. the positively charged peptide and the targeting-delivery peptide) is expressed from a single polynucleotide sequence. The coding sequences of the peptides may be combined in any way to form a single polynucleotide sequence encoding the chimera.

Examples of preferred chimeras for use in the invention are shown in Table 3.

TABLE 3
Preferred chimeras for use in the invention.

| | | SEQ |
|---|---|---|
| Name | Sequence | ID NO: |
| RXR4MSP | RXRRXRRXRRXRXBASSLNIAXC | 50 |
| Pip5eMSP | RXRRBRRXRILFQYRXRBRXRBASSLNIAXC | 51 |
| RXB-MSP-RXB | RXRRBRRXRASSLNIARXRBRXRBC | 52 |
| B-MSP | RXRRBRRXRRBRXBASSLNIAX | 53 |

The biologically active compound is covalently or non-covalently attached to the chimeric cell delivery peptide. Again, this can be done using any method known in the art. Preferably, the cell delivery peptide is attached to the biologically active compound by means of a disulphide bridge or an AEEA (2 aminoethoxy-2-ethoxy acetic acid) linker. The attachment may be by means of an amide linker (preferably a stable amide linker) or a thiol maleimide linker, or an oxime linker or a thioether linker.

### Functional Derivatives

It will be understood that functional derivatives of the specific peptides disclosed herein could be used. Such derivatives are typically peptides that have sequences which have homology to the original peptides. The derivatives may represent fragments of the original peptides or homologues, or may represent peptides that include insertions (amino acid additions) to the original peptides, homologues or said fragments. Typically the derivative has at least 70%, 80% or 90% of the number of amino acids present in the original peptide or may have less than 200% or 150% of the number of amino acids present in the original peptide. The derivative is generally able to enhance the delivery of a compound to a cell, for example as determined by any assay mentioned herein.

### Biologically Active Compounds

A biologically active compound comprised within the constructs of the invention is any compound that may exert a biological effect within a biological cell, typically affecting the expression of one or more genes in the cell. Examples of biologically active compounds include nucleic acids, peptides, proteins, DNAzymes, Ribozymes, chromophores, fluorophores and pharmaceuticals.

Such nucleic acids may be single or double stranded. Single-stranded nucleic acids include those with phosphodiester, 2′O-methyl, 2′ methoxy-ethyl, phosphoramidate, methylphosphonate, and/or phosphorothioate backbone chemistry, peptide nucleic acid (PNA), phosphorodiamidate morpholino oligonucleotide (PMO), locked nucleic acid (LNA), glycol nucleic acid (GNA) and threose nucleic acid (TNA). Double-stranded nucleic acids include plasmid DNA and small interfering RNAs (siRNAs).

The biologically active compound to be delivered is chosen on the basis of the desired effect of that compound on the cell into which it is delivered and the mechanism by which that effect is to be carried out. For example, the compound may be used to treat a disease state within that cell, for example by attenuating the propagation of a pathogen (e.g. a virus), typically by using a small-molecule inhibitor, or by correcting the expression of an aberrantly expressed protein, typically using an anti-sense oligonucleotide (AO) to modulate pre-mRNA splicing (see below). The compound may also be used to diagnose a disease state within that cell, for example by delivering to that cell a compound used to detect a diagnostic marker.

The skeletal muscle disease to be treated may be a muscular dystrophy phenotype, optionally Duchenne muscular dystrophy (DMD), Becker muscular dystrophy, myotonic dystrophy (MD), spinal muscular atrophy, limb-girdle muscular dystrophy (LGMD), facioscapulohumeral muscular dystrophy, congenital muscular dystrophy, oculpharyngeal muscular dystrophy (OMD), distal muscular dystrophy and Emery-Dreifuss muscular dystrophy (EDMD).

Genes implicated in the pathogenesis of these diseases include dystrophin (Duchenne muscular dystrophy and Becker muscular dystrophy), DMPK (DM1 type MD), ZNF9 (DM2 type MD), PABPN1 (OMD), emerin, lamin A or lamin C (EDMD), myotilin (LGMD-1A), lamin A/C (LGMD-1B), caveolin-3 (LGMD-1C), calpain-3 (LGMD-2A), dysferlin (LGMD-2B and Miyoshi myopathy), gamma-sarcoglycan LGMD-2C), alpha-sarcoglycan (LGMD-2D), betaa-sarcoglycan (LGMD-2E), delta-sarcoglycan (LGMD-2F and CMD1L), telethonin (LGMD-2G), TRIM32 (LGMD-2H), fukutin-related protein (LGMD-21), titin (LGMD-2J), and O-mannosyltransferase-1 (LGMD-2K).

The cardiac muscle disease to be treated may be coronary heart disease, congenital heart disease, ischemic, hypertensive, inflammatory or intrinsic cardiomyopathy. Intrinsic cardiomyopathy includes the following disorders (with associated genes): dilated cardiomyopathy (dystrophin, G4.5, actin, desmin, delta-sarcoglycan, troponin T, beta-myosin heavy chain, alpha-tropomyosin, mitochondrial respiratory chain), dilated cardiomyopathy with conduction disease (lamin A/C), hypertrophic cardiomyopathy (beta-myosin heavy chain, troponin T, troponin I, alpha-tropomyosin, myosin-binding protein C, myosin essential light chain, myosin regulatory light chain, titin), hypertrophic cardiomyopathy with Wolff-Parkinson-White syndrome (AMPK, mitochondrial respiratory chain), and left ventricular noncompaction (G4.5, alpha-dystrobrevin).

In one embodiment the biologically active compound is not RNA. In another embodiment the biologically active compound is not siRNA. In one embodiment the targeting-delivery peptide is not TAT peptide.

### Modulation of Pre-RNA Splicing

DNA sequences are transcribed into pre-mRNAs which contain coding regions (exons) and generally also contain intervening non-coding regions (introns). Introns are removed from pre-mRNAs in a precise process called cis-splicing. Splicing takes place as a coordinated interaction of several small nuclear ribonucleoprotein particles (snRNPs) and many protein factors that assemble to form an enzymatic complex known as the spliceosome. Specific motifs in the pre-mRNA that are involved in the splicing process include splice site acceptors, splice site donors, exonic splicing enhancers (ESEs) and exon splicing silencers.

Pre-mRNA can be subject to various splicing events. Alternative splicing can result in several different mRNAs being capable of being produced from the same pre-mRNA. Alternative splicing can also occur through a mutation in the pre-mRNA, for instance generating an additional splice acceptor and/or splice donor sequence (cryptic sequences). Restructuring the exons in the pre-mRNA, by inducing exon skipping or inclusion, represents a means of correcting the expression from pre-mRNA exhibiting undesirable splicing or expression in an individual. Exon restructuring can be used to promote the production of a functional protein in a cell. Restructuring can lead to the generation of a coding region for a functional protein. This can be used to restore an open reading frame that was lost as a result of a mutation.

Antisense oligonucleotides (AOs) can be used to alter pre-mRNA processing via the targeted blockage of motifs involved in splicing. Hybridisation of antisense oligonucletides to splice site motifs prevents normal spliceosome assembly and results in the failure of the splicing machinery to recognize and include the target exon(s) in the mature gene transcript. This approach can be applied to diseases caused by aberrant splicing, or where alteration of normal splicing would abrogate the disease-causing mutation. This includes: (i) blockage of cryptic splice sites, (ii) exon removal or inclusion to alter isoform expression, and (iii) removal of exons to either eliminate a nonsense mutation or restore the reading frame around a genomic deletion.

An example of a gene in which the reading frame may be restored is the Duchenne muscular dystrophy (DMD) gene. The dystrophin protein is encoded by a plurality of exons over a range of at least 2.6 Mb. DMD is mainly caused by nonsense and frame-shift mutations in the dystrophin gene resulting in a deficiency in the expression of dystrophin protein. The dystrophin protein consists of two essential functional domains connected by a central rod domain. Dystrophin links the cytoskeleton to the extracellular matrix and is thought to be required to maintain muscle fibre stability during contraction. Mutations that disrupt the open reading frame result in prematurely truncated proteins unable to fulfill their bridge function. Ultimately this leads to muscle fibre damage and the continuous loss of muscle fibres, replacement of muscle tissue by fat and fibrotic tissue, impaired muscle function, and eventually the severe phenotype observed for DMD patients. In contrast, mutations that maintain the open reading frame allow for the generation of internally deleted, but partially functional, dystrophins. These mutations are associated with Becker muscular dystrophy (BMD), a much milder disease when compared with DMD. Patients generally remain ambulant until later in life and have near normal life expectancies.

The inventors have discovered that AOs based on peptide nucleic acid (PNAs) that are capable of targeting splice site motifs in mutated dystrophin mRNA can efficiently induce exon skipping. It is possible to target an exon which flanks an out-of frame deletion or duplication so that the reading frame can be restored and dystrophin production allowed. The removal of the mutated exon in this way allows shortened but functional (BMD-like) amounts of dystrophin protein to be produced. As a result, a severe DMD phenotype can be converted into a milder BMD phenotype.

Dystrophia myotonica (myotonic dystrophy) type 1 (DM1), the most common muscular dystrophy affecting adults, is caused by expansion of a CTG repeat in the 3′ untranslated region of the gene encoding the DM protein kinase (DMPK). Evidence suggests that DM1 is not caused by abnormal expression of DMPK protein, but rather that it involves a toxic gain of function by mutant DMPK transcripts that contain an expanded CUG repeat (CUG^{exp}). The transcripts containing a CUG^{exp} tract elicit abnormal regulation of alternative splicing, or spliceopathy. The splicing defect, which selectively affects a specific group of pre-mRNAs, is thought to result from reduced activity of splicing factors in the muscleblind (MBNL) family, increased levels of CUG-binding protein 1, or both. Myotonia in mouse models of DM appears to result from abnormal inclusion of exon 7a in the ClC-1 mRNA. Inclusion of exon 7a causes frame shift and introduction of a premature termination codon in the ClC-1 mRNA. A therapeutic strategy for myotonic dystrophy is therefore to repress the inclusion of exon 7a in the mouse ClC-1 mRNA, or the corresponding exon in human ClC-1 mRNA.

Just as targeted blockage of consensus splice sites and ESEs promotes exon exclusion, the blockage of exonic or intronic splicing silencers, or the introduction of splicing enhancer sequences, can enhance exon inclusion. This offers the ability to enhance expression of alternatively spliced ‘weak’ exons to induce the most functionally preferable isoform. In spinal muscular atrophy (SMA), mutations in the survival motor neuron (SMN1) gene are responsible for a degenerative disease that presents as childhood muscle weakness and, in the more serious forms, can cause fatal respiratory failure. The severity of the disease is modified by the production of SMN protein encoded by the paralogous gene, SMN2. Although SMN2 is nearly identical to SMN1, a silent C to T mutation in exon 7 abrogates an ESE site, weakening recognition of the upstream 3′ splice site and resulting in the majority of SMN2 transcripts lacking exon 7. As this SMNΔ7 isoform is unstable, and at best, only partially functional, the level of full-length SMN protein is an important modifier of patient disease severity. Antisense technology can therefore be used to promote exon 7 inclusion in the SMN2 transcript.

In a preferred embodiment of the invention the construct comprises an antisense-based system, for example comprising PNA or PMO, for inducing the skipping or inclusion of one or more exons in a pre-mRNA, thereby resulting in the expression of functional protein. Accordingly, disclosed is a method of correcting expression of a gene in a human cell having a muscle disease or muscular dystrophy phenotype, wherein without correction the gene fails to express functional protein due to one or more mutations, said method comprising delivering to the cell a nucleic acid comprising a sequence capable of targeting a sequence responsible for exon skipping in the mutated pre-mRNA at an exon to be skipped or included, wherein expression is corrected by the PNA inducing exon skipping or inclusion and thereby correcting the expression of said mutated pre-mRNA

The muscle disease or muscular dystrophy may be any muscular disease or dystrophy that is caused by the aberrant expression of a protein. The aberrant protein expression may be as a result of one or more nonsense or frame-shift mutations. The aberrant protein expression may be the result of a mutation that weakens a splice site resulting in the inclusion of an undesirable exon. Alternatively, the mutation may introduce a cryptic splice site resulting in the splicing of an exon that is desired to be included for protein function.

Examples of muscle diseases include Duchenne muscular dystrophy (DMD), myotonic dystrophy, spinal muscular atrophy, limb-girdle muscular dystrophy, facioscapulohumeral muscular dystrophy, congenital muscular dystrophy, oculpharyngeal muscular dystrophy, distal muscular dystrophy and Emery-Dreifuss dystrophy. Where the disease is DMD, the gene for which expression may be corrected is the dystrophin gene. Where the disease is myotonic dystrophy, the gene for which expression may be corrected is the muscle specific chloride channel (ClC-1) gene. Where is disease is spinal muscular atrophy, the gene for which expression may be corrected is the SMN2 gene.

The human cell may be any human cell in which the gene for which expression is to be corrected has one or more mutations. The one or more mutations may be nonsense or frame-shift mutations. The one or more mutations may strengthen a cryptic splice site or may weaken a splice site. The cell has a muscle disease/dystrophy phenotype, i.e. does not produce a particular functional protein. The cell may be taken from a human patient that has a muscle disease/dystrophy. For example, the cell may be taken from a human patient that has DMD, myotonic dystrophy or spinal muscular atrophy.

Nucleic acid such as PNA or PMO can be used for the purpose of inducing exon skipping, or alternatively, exon inclusion. More than one exon can be induced to be skipped at a time. This is desirable because there are often numerous exons in a gene that could potentially be mutated resulting in muscle disease/dystrophy. By targeting the skipping of more than one exon it is possible to remove a larger region of potentially mutant mRNA resulting in the expression of a shortened but functional protein. Any number of exons may be skipped provided that the remaining exons are sufficient to result in the expression of suitably functional protein. Accordingly, 1, 2, 3, 4, 5, 6, 7, 8 or more exons may be skipped.

The disclosed method results in the induction of expression of functional protein. Typically, the amount of functional protein expressed in the cell is at least 10% of the amount of functional protein expressed in a cell in which the gene is not mutated. Preferably, the amount of functional protein expressed in a cell is at least 15%, 20%, 25%, 30%, or more preferably, at least 40% or 50% of the amount of functional protein expressed in a cell in which the gene is not mutated. A method for determining the relative amount of functional protein expressed may be any suitable method known in the art, for example Western blotting.

The functional protein that is expressed by the method is preferably capable of performing the function(s) of the corresponding protein expression from a non-mutated gene. The functional protein may not be 100% as effective as the normal protein but is preferably at least 50%, 60%, 70%, 80%, 90% or more preferably, at least 95% as effective as the normal protein. Functional activity may be determined by any method known in the art to the skilled person that is relevant to the protein concerned.

### Therapeutic Treatment

The ability of the constructs of the invention to deliver biologically active compounds to cells, e.g. cardiac and skeletal muscle cells, results in the suitability of the constructs of the invention for therapeutic treatment of disease, such as muscle disease or muscular dystrophy, in a subject having such a disease. As used herein, the term “treatment” is meant to encompass therapeutic, palliative and prophylactic uses.

This method of treatment or diagnosis is suitable for any patient that has, may have, or is suspected of having, a disease, such as a muscle disease or muscular dystrophy. The disease may be caused by a nonsense or frameshift mutation. The aberrant protein expression may be the result of a mutation that weakens a splice site resulting in the inclusion of an unsuitable exon. Alternatively, the mutation may introduce a cryptic splice site resulting in the splicing of an exon that is important for protein function. The muscle disease or muscular dystrophy may be any muscle disease or dystrophy. Examples include Duchenne muscular dystrophy (DMD), myotonic dystrophy and spinal muscular atrophy.

Symptoms of DMD which may be used to determine whether a subject has DMD include progressive muscle wasting (loss of muscle mass), poor balance, frequent falls, walking difficulty, waddling gait, calf pain, limited range movement, muscle contractures, respiratory difficulty, drooping eyelids (ptosis), gonadal atrophy and scoliosis (curvature of the spine). Other symptoms can include cardiomyopathy and arrhythmias.

Symptoms of myotonic dystrophy which may be used to determine whether a subject has myotonic dystrophy include abnormal stiffness of muscles and myotonia (difficulty or inability to relax muscles). Other symptoms of myotonic dystrophy include weakening and wasting of muscles (where the muscles shrink over time), cataracts, and heart problems. Myotonic dystrophy affects heart muscle, causing irregularities in the heartbeat. It also affects the muscles of the digestive system, causing constipation and other digestive problems. Myotonic dystrophy may cause cataracts, retinal degeneration, low IQ, frontal balding, skin disorders, atrophy of the testicles, insulin resistance and sleep apnea.

A muscle disease of muscular dystrophy may be diagnosed on the basis of symptoms and characteristic traits such as those described above and/or on the results of a muscle biopsy, DNA or blood test. Blood tests work by determining the level of creatine phosphokinase (CPK). Other tests may include serum CPK, electromyography and electrocardiography. Muscular dystrophies can also alter the levels of myoglobin, LDH, creatine, AST and aldolase.

The method of treatment or diagnosis can be used to treat a subject of any age. The subject is preferably mammal, such as human. Preferably an individual to be treated or diagnosed is as young as possible and/or before symptoms of the disease or condition develop. For example, it is preferable to treat an individual before muscle damage occurs in order to preserve as much muscle as possible. For example, the age of onset of DMD is usually between 2 and 5 years old. Without treatment, most DMD sufferers die by their early twenties, typically from respiratory disorders. Typically therefore, the age of the subject to be treated for DMD is from 2 to 20 years old. More preferably, the age of the subject to be treated is from 4 to 18, from 5 to 15 or from 8 to 12. Myotonic dystrophy generally affects adults with an age at onset of about 20 to about 40 years. Typically, the age of the subject to be treated for myotonic dystrophy is from 2 to 40 years old. More preferably, the age of the subject to be treated is from 4 to 35, from 8 to 30 or from 12 to 25. Preferably the individual to be treated is asymptomatic.

The constructs of the invention may be used to deliver biologically active compounds into any type of muscle tissue. The target muscle tissue may be skeletal muscle, cardiac muscle, or smooth muscle. In DMD patients, targeting the heart muscle may be preferable in patients with cardiac disease or early cardiac symptoms. Such patients may be preferable to treat because of the early mortality associated with this component of the disease.

Current medications and treatments for muscular dystrophy are limited. Inactivity can worsen the disease. Physical therapy and orthopaedic instruments may be helpful. The cardiac problems that occur with myotonic dystrophy and Emery-Dreifuss muscular dystrophy may require a pacemaker. Conventional methods of coping with the disease include exercise, drugs that slow down or eliminate muscle wasting, anabolic steroids and dietary supplements such as creatine and glutamine. The anti-inflammatory corticosteroid prednisone may be used to improve muscle strength and delay the progression of the disease. Other nutritional supplements and steroids that may be used in the treatment of DMD include deflazacort, albuterol, creatine, anabolic steroids, and calcium blockers. The myotonia occurring in myotonic dystrophy may be treated with medications such as quinine, phenyloin or mexiletine. All of the above treatments are aimed at slowing down the progression of the disease or reducing its symptoms. The treatment of the invention may be administered in combination with any such form of treating or alleviating the symptoms of muscle disease or muscular dystrophy.

### Nucleic Acids, Peptide Nucleic Acid (PNA) and Phosphorodiamidate Morpholino Oligonucleotides (PMO)

In PNAs, the sugar phosphate backbone of DNA is replaced by an achiral polyamide backbone. PNAs have a high affinity for DNA and RNA and high sequence specificity. They are also highly resistant to degradation, being protease- and nuclease-resistant. PNAs are also stable over a wide pH range. Similarly, in PMOs, the sugar phosphate backbone is replaced by a phosphorodiamidate morpholino backbone. These are also highly resistant to degradation, being protease- and nuclease-resistant and have a high affinity for RNA and high sequence specificity.

The nucleic acids (such as PNAs and PMOs) used in the invention are typically at least 10 bases long, such as at least 12, 14, 15, 18, 20, 23 or 25 or more bases in length. Typically, the nucleic acid is less than 35 bases in length. Such as less than 34, 32, 30 or 28 bases long. Preferably, the nucleic acid will be in the range of 15 to 30 bases long, more preferably 15 to 25 or 20 to 30 bases long. The nucleic acids may be 18 or 25 bases in length.

The AOs are complementary to and selectively hybridise to one or more sequences that are responsible for or contribute to the promotion of exon splicing or inclusion. Such a sequence may be a splice site donor sequence, splice site acceptor sequence, splice site enhancer sequence or splice site silencer sequence. Splice site donor, acceptor and enhancer sequences are involved in the promotion of exon splicing and therefore can be targeting with one or more AOs in order to inhibit exon splicing. Splice site silencers are involved in inhibiting splicing and can therefore be targeted with AOs in order to promote exon splicing.

Splice site donor, acceptor, enhancer and silencer sequences may be located within the vicinity of the 5′ or 3′ end of the exon to be spliced from or, in the case of silencer sequences, included into the final mRNA. Splice site acceptor or donor sequences and splice site enhancer or silencer sequences are either known in the art or can be readily determined. Bioinformatic prediction programmes can be used to identify gene regions of relevance to splicing events as a first approximation. For example, software packages such as RESCUE-ESE, ESEfinder, and the PESX server predict putative ESE sites. Subsequent empirical experimental work, using splicing assays well known in the art, can then be carried out in order to validate or optimise the sequences involved in splicing for each exon that is being targeted.

Any exon in which there is a non-sense or frame-shift inducing mutation may be a potential target for deletion from the pre-mRNA by exon skipping. Any of the exons in the dystrophin gene can be targeted for deletion from the dystrophin pre-mRNA. Preferably, the exons that are targeted for deletion are any of the exons in the human dystrophin gene except for exons 65 to 69, which are essential for protein function. Preferably the exon(s) to be deleted are those that are commonly mutated in DMD, i.e. any of exons 2 to 20 or exons 45 to 53.

Preferably, the patient is tested for which mutation they have in order to determine which exon is to be deleted or included. Preferably, the sequence of the nucleic acid used for exon skipping comprises a sequence that is capable of selectively hybridising to a sequence that spans the exon/intron boundary of the exon to be deleted or included. The exon/intron boundary may be the 3′ or 5′ boundary of the exon to be included or deleted. The exon/intron boundary sequence information for a particular gene may be obtained from any source of sequence information, such as the ensemble database. Sequence information, including the exon/intron boundary locations, for the human and mouse dystrophin genes may be found at the following web links:

Human: http://vega.sanger.ac.uk/Homo_sapiens/transview?transcript=OTTHUMT00000056182
Mouse: http://vega.sanger.ac.uk/Mus_musculus/transview?transcript=OTTMUST00000043357

The currently known mutations, including point mutations, deletions duplications in the entire human dystrophin gene may be accessed at the following web link: http://www.dmd.nl/DMD_deldup.html

More preferably, the AO sequence is selected from sequences capable of selectively hybridising to the exon/intron boundary sequences provided in Table 4 or homologues thereof. The nomenclature in Table 4 is based upon target species (H, human, M, mouse), exon number, and annealing coordinates as described by Mann et al 2002 (Journal of Gene Medicine, 4: 644-654). The number of exonic nucleotides from the acceptor site is indicated as a positive number, whereas intronic bases are given a negative value. For example, H16A(−06+25) refers to an antisense oligonucleotide for human dystrophin exon 16 acceptor region, at coordinates 6 intronic bases from the splice site to 25 exonic bases into exon 16. The total length of this AO is 31 nucleotides and it covers the exon 16 acceptor site.

TABLE 4
Sequences of exon/intron boundaries in human and mouse
dystrophin pre-mRNA (SEQ ID NO: 55 to 175).

| Nomenclature | Sequence (5′-3′) |
|---|---|
| H2A(+12+41) | CCA UUU UGU GAA UGU UUU CUU UUG AAC AUC |
| H3A(+20+40) | GUA GGU CAC UGA AGA GGU UCU |
| H4A(+11+40) | UGU UCA GGG CAU GAA CUC UUG UGG AUC CUU |
| H5A(+25+55) | UCA GUU UAU GAU UUC CAU CUA CGA UGU CAG U |
| H6A(+69+91) | UAC GAG UUG AUU GUC GGA CCC AG |
| H7A(+45+67) | UGC AUG UUC CAG UCG UUG UGU GG |
| H9A(−06+23) | CCC UGU GCU AGA CUG ACC GUG AUC UGC AG |
| H12A(+52+75) | UCU UCU GUU UUU GUU AGC CAG UCA |
| H13A(+77+100) | CAG CAG UUG CGU GAU CUC CAC UAG |
| H14A(+32+61) | GUA AAA GAA CCC AGC GGU CUU CUG UCC AUC |
| H15A(+48+71) | UCU UUA AAG CCA GUU GUG UGA AUC |
| H16A(−12+19) | CUA GAU CCG CUU UUA AAA CCU GUU AAA ACA A |
| H18A(+24+53) | CAG CUU CUG AGC GAG UAA UCC AGC UGU GAA |
| HM19A(+35+65) | GCC UGA GCU GAU CUG CUG GCA UCU UGC AGU U |
| H22A(+125+146) | CUG CAA UUC CCC GAG UCU CUG C |
| H23A(+69+98) | CGG CUA AUU UCA GAG GGC GCU UUC UUC GAC |
| H24A(+51+73) | CAA GGG CAG GCC AUU CCU CCU UC |
| H25A(+95+119) | UUG AGU UCU GUC UCA AGU CUC GAA G |
| H27A(+82+106) | UUA AGG CCU CUU GUG CUA CAG GUG G |
| H28A(+99+124) | CAG AGA UUU CCU CAG CUC CGC CAG GA |
| H29A(+57+81) | UCC GCC AUC UGU UAG GGU CUG UGC C |
| H30A(+25+50) | UCC UGG GCA GAC UGG AUG CUC UGU UC |
| H31D(+03−22) | UAG UUU CUG AAA UAA CAU AUA CCU G |
| H32A(44+73) | CUU GUA GAC GCU GCU CAA AAU UGG CUG GUU |
| H33A(+64+88) | CCG UCU GCU UUU UCU GUA CAA UCU G |
| H35A(+24+53) | UCU GUG AUA CUC UUC AGG UGC ACC UUC UGU |
| H37A(+134+157) | UUC UGU GUG AAA UGG CUG CAA AUC |
| H38A(+88+112) | UGA AGU CUU CCU CUU UCA GAU UCA C |
| H39A(+62+91) | UUU CCU CUC GCU UUC UCU CAU CUG UGA UUC |
| H41A(+44+69) | CAA GCC CUC AGC UUG CCU ACG CAC UG |
| H42A(−4+23) | AUC GUU UCU UCA CGG ACA GUG UGC UGG |
| H47A(−06+24) | CAG GGG CAA CUC UUC CAC CAG UAA CUG AAA |
| H49A(−11+16) | CUG CUA UUU CAG UUU CCU GGG GAA AAG |
| H51A(+66+90) | ACA UCA AGG AAG AUG GCA UUU CUA G |
| H52A(+12+41) | UCC AAC UGG GGA CGC CUC UGU UCC AAA UCC |
| H53A(+39+69) | CAU UCA ACU GUU GCC UCC GGU UCU GAA GGU G |
| H72A(+02+28) | GUG UGA AAG CUG AGG GGA CGA GGC AGG |
| H74A(+48+72) | CGA GGC UGG CUC AGG GGG GAG UCC U |
| H75A(+34+58) | GGA CAG GCC UUU AUG UUC GUG CUG C |
| H77A(+16+42) | CUG UGC UUG UGU CCU GGG GAG GAC UGA |
| H78A(+04+29) | UCU CAU UGG CUU UCC AGG GGU AUU UC |
| H11A(+75+97) | CAU CUU CUG AUA AUU UUC CUG UU |
| H21A(+86+108) | GUC UGC AUC CAG GAA CAU GGG UC |
| H36A(+22+51) | UGU GAU GUG GUC CAC AUU CUG GUC AAA AGU |
| H40A(−5+17) | CUU UGA GAC CUC AAA UCC UGU U |
| H43A(+101+120) | GGA GAG AGC UUC CUG UAG CU |
| H44A(+61+84) | UGU UCA GCU UCU GUU AGC CAC UGA |
| H46A(+107+137) | CAA GCU UUU CUU UUA GUU GCU GCU CUU UUC C |
| H48A(−07+23) | UUC UCA GGU AAA GCU CUG GAA ACC UGA AAG |
| H57A(−12+18) | CUG GCU UCC AAA UGG GAC CUG AAA AAG AAC |
| H60A(+37+66) | CUG GCG AGC AAG GUC CUU GAC GUG GCU CAC |
| H61A(+10+40) | GGG CUU CAU GCA GCU GCC UGA CUC GGU CCU C |
| H68A(+22+48) | CAU CCA GUC UAG GAA GAG GGC CGC UUC |
| H70A(+98+121) | CCU CUA AGA CAG UCU GCA CUG GCA |
| H71A(−03+21) | AAG UUG AUC AGA GUA ACG GGA CUG |
| H73A(+06+30) | GAU CCA UUG CUG UUU UCC AUU UCU G |
| H26A(−07+19) | CCU CCU UUC UGG CAU AGA CCU UCC AC |
| H45A(−06+20) | CCA AUG CCA UCC UGG AGU UCC UGU AA |
| H50A(+02+30) | CCA CUC AGA GCU CAG AUC UUC UAA CUU CC |
| H55A(+141+160) | CUU GGA GUC UUC UAG GAG CC |
| H56A(+102+126) | GUU AUC CAA ACG UCU UUG UAA CAG G |
| H58A(+21+45) | ACU CAU GAU UAC ACG UUC UUU AGU U |
| H59A(−06+16) | UCC UCA GGA GGC AGC UCU AAA U |
| H62A(+8+34) | GAG AUG GCU CUC UCC CAG GGA CCC UGG |
| H63A(+11+35) | UGG GAU GGU CCC AGC AAG UUG UUU G |
| H64A(+47+74) | GCA AAG GGC CUU CUG CAG UCU UCG GAG |
| H66A(−8+19) | GAU CCU CCC UGU UCG UCC CCU AUU AUG |
| H67A(+22+47) | GCG CUG GUC ACA AAA UCC UGU UGA AC |
| H69A(−06+18) | UGC UUU AGA CUC CUG UAC CUG AUA |
| H76A(+53+79) | GCU GAC UGC UGU CGG ACC UCU GUA GAG |
| H8A(−06+18) | GAU AGG UGG UAU CAA CAU CUG UAA |
| H10A(−05+16) | CAG GAG CUU CCA AAU GCU GCA |
| H10A(+98+119) | UCC UCA GCA GAA AGA AGC CAC G |
| H17A(−07+16) | UGA CAG CCU GUG AAA UCU GUG AG |
| H20A(+44+71) | CUG GCA GAA UUC GAU CCA CCG GCU GUU C |
| H20A(147+168) | CAG CAG UAG UUG UCA UCU GCU C |
| H34A(+46+70) | CAU UCA UUU CCU UUC GCA UCU UAC G |
| H34A(+95+120) | AUC UCU UUG UCA AUU CCA UAU CUG UA |
| H54A(+67+89) | UCU GCA GAA UAA UCC CGG AGA AG |
| H65A(−11+14) | GCU CAA GAG AUC CAC UGC AAA AAA C |
| H65A(+63+87) | UCU GCA GGA UAU CCA UGG GCU GGU C |
| H65D(+15−11) | GCC AUA CGU ACG UAU CAU AAA CAU UC |
| H16A(−17+08) | UUU AAA ACC UGU UAA AAC AAG AAA G |
| H16A(−12+19) | CUA GAU CCG CUU UUA AAA CCU GUU AAA ACA A |
| H16A(−06+19) | CUA GAU CCG CUU UUA AAA CCU GUU A |
| H16A(−06+25) | UCU UUU CUA GAU CCG CUU UUA AAA CCU GUU A |
| H16A(−07+13) | CCG CUU UUA AAA CCU GUU AA |
| H16A(+01+25) | UCU UUU CUA GAU CCG CUU UUA AAA C |
| H16A(+06+30) | CUU UUU CUU UUC UAG AUC CGC UUU U |
| H16A(+11+35) | GAU UGC UUU UUC UUU UCU AGA UCC G |
| H16A(+12+37) | UGG AUU GCU UUU UCU UUU CUA GAU CC |
| H16A(+45+67) | GAU CUU GUU UGA GUG AAU ACA GU |
| H16A(+87+109) | CCG UCU UCU GGG UCA CUG ACU UA |
| H16A(+92+116) | CAU GCU UCC GUC UUC UGG GUC ACU G |
| H16A(+105+126) | GUU AUC CAG CCA UGC UUC CGU C |
| H16D(+11−11) | GUA UCA CUA ACC UGU GCU GUA C |
| H16D(+05−20) | UGA UAA UUG GUA UCA CUA ACC UGU G |
| H46A(+107+137) | CAA GCU UUU CUU UUA GUU GCU GCU CUU UUC C |
| H51A(−01+25) | ACC AGA GUA ACA GUC UGA GUA GGA GC |
| H51A(+61+90) | ACA UCA AGG AAG AUG GCA UUU CUA GUU UGG |
| H51A(+66+90) | ACA UCA AGG AAG AUG GCA UUU CUA G |
| H51A(+66+95) | CUC CAA CAU CAA GGA AGA UGG CAU UUC UAG |
| H51A(+111+134) | UUC UGU CCA AGC CCG GUU GAA AUC |
| H51A(+175+195) | CAC CCA CCA UCA CCC UCU GUG |
| H51A(+199+220) | AUC AUC UCG UUG AUA UCC UCA A |
| H51D(+08−17) | AUC AUU UUU UCU CAU ACC UUC UGC U |
| H51D(+16−07) | CUC AUA CCU UCU GCU UGA UGA UC |
| H53A(−07+18) | GAU UCU GAA UUC UUU CAA CUA GAA U |
| H53A(−12+10) | AUU CUU UCA ACU AGA AUA AAA G |
| H53A(+23+47) | CTG AAG GTG TTC TTG TAC TTC ATC C |
| H53A(+39+62) | CUG UUG CCU CCG GUU CUG AAG GUG |
| H53A(+39+69) | CAU UCA ACU GUU GCC UCC GGU UCU GAA GGU G |
| H53A(+45+69) | CAU UCA ACU GUU GCC UCC GGU UCU G |
| H53A(+124+145) | UUG GCU CUG GCC UGU CCU AAG A |
| H53A(+151+175) | GUA UAG GGA CCC UCC UUC CAU GAO U |
| H53D(+09−18) | GGU AUC UUU GAU ACU AAC CUU GGU UUC |
| H53D(+14−07) | UAC UAA CCU UGG UUU CUG UGA |
| M23D(+07−18) | GGC CAA ACC UCG GCU UAC CUG AAA U |
| M23D(+02−18) | GGC CAA ACC UCG GCU UAC CU |
| M23D(+12−18) | GGC CAA ACC UCG GCU UAC CUG AAA UUU UCG |
| M23D(+07−23) | UUA AAG GCC AAA CCU CGC CUU ACC UGA AAU |

Examples of preferred AO sequences capable of inducing the splicing of exon 7a in the mouse ClC-1 gene are sequences capable of selectively hybridising to the 3′ or 5′ splice sites of exon 7a. Such preferred AO sequences may be capable of specifically hybridising to a sequence in Table 5 or a homologue thereof.

TABLE 5
Sequences of exon/intron boundaries in the
mouse CIC-1 pre-mRNA for mouse exon 7a
(SEQ ID NO: 176 and 177).

| Nomenclature | Sequence (5′-3′) |
|---|---|
| M7a(−17+14) | GUG CUU CUC UGU UGC AGA CCG UGC |
| | CUG GGC A |
| M7a(+13−18) | GCC CCT GAU GGA GGC AAG UUU CAC |
| | UUC CUC C |

Typically, only one AO sequence is used to induce or inhibit exon skipping in a cell. However, more than one different AO can be delivered to the sample of human cells or a patient, e.g. a cocktail of 2, 3, 4 or 5 or more different AO sequences can be used to drive exon skipping or inhibit exon skipping in a cell. Such a combination of different AO sequences can be delivered simultaneously, separately or sequentially.

Selective hybridisation means that generally the polynucleotide can hybridize to the relevant polynucleotide, or portion thereof, at a level significantly above background. The signal level generated by the interaction between the polynucleotides is typically at least 10 fold, preferably at least 100 fold, as intense as interactions between other polynucleotides. The intensity of interaction may be measured, for example, by radiolabelling the polynucleotide, e.g. with ³²P. Selective hybridisation is typically achieved using conditions of medium to high stringency (for example 0.03M sodium chloride and 0.003M sodium citrate at from about 50° C. to about 60° C.).

PNAs are produced synthetically using any known technique in the art. PNA is a DNA analogue in which a polyamide backbone replaces the normal phosphate and deoxyribose ring of DNA. Despite a radical change to the natural structure, PNA is capable of sequence-specific binding to DNA or RNA. Characteristics of PNA include a high binding affinity to complementary DNA or RNA, a destabilizing effect caused by single-base mismatch, resistance to nucleases and proteases, hybridization with DNA independent of salt concentration and triplex formation with homopurine DNA.

PNAs may be obtained commercially from Panagene and may contain functionalities such as a cysteine residue or bromoaceytl group suitable for joining to a peptide. Panagene™ has developed its proprietary Bts PNA monomers (Bts; benzothiazole-2-sulfonyl group) and proprietary oligomerisation process. The PNA oligomerisation using Bts PNA monomers is composed of repetitive cycles of deprotection, coupling and capping. Panagene's patents to this technology include U.S. Pat. No. 6,969,766, U.S. Pat. No. 7,211,668, U.S. Pat. No. 7,022,851, U.S. Pat. No. 7,125,994, U.S. Pat. No. 7,145,006 and U.S. Pat. No. 7,179,896. Methods for preparation of peptide-PNA conjugates are disclosed in Turner et al (2005) Nucleic Acids Res., 33, 6837-6849 and in Ivanova et al. (2008) Nucleic Acids Res. 36, 6418-6428.

PMOs are produced synthetically using any known technique in the art. PMO is a DNA analogue in which a phosphorodiamidate morpholino backbome replaces the normal phosphate and deoxyribose ring of DNA. Characteristics of PMO include a high binding affinity to complementary DNA or RNA, a destabilizing effect caused by single-base mismatch, resistance to nucleases and proteases and hybridization with DNA independent of salt concentration. PMO may be obtained commercially from Gene Tools LLC and may obtained with a 5′ amino linker suitable for covalent joining to a cell delivery peptide. Methods for conjugation of peptides to PMO are disclosed in Moulton et al (2004), 15, 290-299.

### Delivery Using Glucose Analogues

The invention provides a composition for use in delivering a nucleic acid or a conjugate of the invention to a cell. The conjugate may be any of the conjugates mentioned herein, and in one embodiment the conjugate does not comprise a nucleic acid (but comprises another type of biologically active compound instead). The composition comprises a glucose analogue, preferably at a concentration of 2 to 50%, such as 4 to 20% or 6 to 15%. The glucose analogue is typically a sugar (excluding glucose), and in certain embodiments may be galactose, mannose, fructose, 2-DG, 3-OMG or AMG.

### Homologues

Homologues of polynucleotide and polypeptide sequences are referred to herein. Such homologues typically have at least 70% homology, preferably at least 80, 90%, 95%, 97% or 99% homology, for example over a region of at least 5, 10, 15, 20, 25 or more contiguous nucleotides or amino acids or over the entire length of the original polynucleotide or polypeptide. The homology may be calculated on the basis of nucleotide or amino acid identity (sometimes referred to as “hard homology”).

For example the UWGCG Package provides the BESTFIT program which can be used to calculate homology (for example used on its default settings) (Devereux et al (1984) Nucleic Acids Research 12, p 387-395). The PILEUP and BLAST algorithms can be used to calculate homology or line up sequences (such as identifying equivalent or corresponding sequences (typically on their default settings), for example as described in Altschul S. F. (1993) J Mol Evol 36:290-300; Altschul, S, Fetal (1990) J Mol Biol 215:403-10.

Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two polynucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The homologous sequence typically differs by at least 1, 2, 5, 10, 20 or more mutations (which may be substitutions, deletions or insertions of nucleotides or amino acids). These mutations may be measured across any of the regions mentioned above in relation to calculating homology.

### Delivery/Administration

The constructs of the invention may be administered by any suitable means. Administration to a human or animal subject may be selected from parenteral, intramuscular, intracerebral, intravascular, subcutaneous, or transdermal administration. Typically the method of delivery is by injection. Preferably the injection is intramuscular or intravascular (e.g. intravenous). A physician will be able to determine the required route of administration for each particular patient.

The constructs are preferably delivered as a composition. The composition may be formulated for parenteral, intramuscular, intracerebral, intravascular (including intravenous), subcutaneous, or transdermal administration. For example, uptake of nucleic acids by mammalian cells is enhanced by several known transfection techniques, for example, those that use transfection agents. The formulation that is administered may contain such agents. Examples of these agents include cationic agents (for example calcium phosphate and DEAE-dextran) and lipofectants (for example lipofectam™ and transfectam™).

Compositions for parenteral administration may include sterile aqueous solutions which may also contain buffers, diluents and other suitable additives. In some cases it may be more effective to treat a patient with a construct of the invention in conjunction with other disease therapeutic modalities (such as those described herein) in order to increase the efficacy of the treatment.

The constructs of the invention may be formulated in a pharmaceutical composition, which may include pharmaceutically acceptable carriers, thickeners, diluents, buffers, preservatives, surface active agents, neutral or cationic lipids, lipid complexes, liposomes, penetration enhancers, carrier compounds and other pharmaceutically acceptable carriers or excipients and the like in addition to the construct. The composition may comprise other active agents that are used in therapy (e.g. anti-inflammatories for DMD therapy).

The constructs may be used in combination with other methods of molecular therapy. For example, the construct may be delivered in combination (simultaneously, separately or sequentially) with a gene or partial gene encoding the protein which is mutated in the individual. For example, the gene may be the full-length or partial sequence of the dystrophin gene in cases of DMD. Gene therapy targeting the myostatin gene or its receptor may also be used in conjunction with the construct(s) in order to increase muscle mass and thereby restore strength in any remaining muscle. Gene delivery may be carried out by any means, but preferably via a viral vector.

Formulations for parenteral administration may include sterile aqueous solutions which may also contain buffers, liposomes, diluents and other suitable additives. Pharmaceutical compositions comprising the construct provided herein may include penetration enhancers in order to enhance the delivery of the construct. Penetration enhancers may be classified as belonging to one of five broad categories, i.e. fatty acids, bile salts, chelating agents, surfactants and non-surfactants. One or more penetration enhancers from one or more of these broad categories may be included.

Various fatty acids and their derivatives which act as penetration enhancers include, for example, oleic acid, lauric acid, capric acid, myristic acid, palmitic acid, stearic acid, linoleic acid, linolenic acid, dicaprate, tricaprate, recinleate, monoolein (1-monooleoyl-rac-glycerol), dilaurin, caprylic acid, arachidonic acid, glyceryl 1-monocaprate, 1-dodecylazacycloheptan-2-one, acylcarnitines, acylcholines, mono- and di-glycerides and physiologically acceptable salts thereof (i.e. oleate, laurate, caprate, myristate, palmitate, stearate, linoleate, etc).

Various natural bile salts, and their synthetic derivatives, act as penetration enhancers. Thus, the term “bile salt” includes any of the naturally occurring components of bile as well as any of their synthetic derivatives.

Complex formulations comprising one or more penetration enhancers may be used. For example, bile salts may be used in combination with fatty acids to make complex formulations. Chelating agents include, but are not limited to, disodium ethylenediaminetetraacetate (EDTA), citric acid, salicylates (e.g. sodium salicylate, 5-methoxysalicylate and homovanilate), N-acyl derivatives of collagen, laureth-9 and N-amino acyl derivatives of beta-diketones (enamines). Chelating agents have the added advantage of also serving as DNase inhibitors.

Surfactants include, for example, sodium lauryl sulfate, polyoxyethylene-9-lauryl ether and polyoxyethylene-20-cetyl ether and perfluorochemical emulsions, such as FC-43. Non-surfactants include, for example, unsaturated cyclic ureas, 1-alkyl- and 1-alkenylazacyclo-alkanone derivatives and non-steroidal anti-inflammatory agents such as diclofenac sodium, indomethacin and phenylbutazone.

A “pharmaceutically acceptable carrier” (excipient) is a pharmaceutically acceptable solvent, suspending agent or any other pharmacologically inert vehicle for delivering one or more nucleic acids to a subject. The pharmaceutically acceptable carrier may be liquid or solid and is selected with the planned manner of administration in mind so as to provide for the desired bulk, consistency etc when combined with a nucleic acid and the other components of a given pharmaceutical composition. Typical pharmaceutically acceptable carriers include, but are not limited to, binding agents (e.g. pregelatinised maize starch, polyvinylpyrrolidone or hydroxypropyl methylcellulose, etc); fillers (e.g. lactose and other sugars, microcrystalline cellulose, pectin, gelatin, calcium sulfate, ethyl cellulose, polyacrylates or calcium hydrogen phosphate, etc); lubricants (e.g. magnesium stearate, talc, silica, colloidal silicon dioxide, stearic acid, metallic stearates, hydrogenated vegetable oils, corn starch, polyethylene glycols, sodium benzoate, sodium acetate, etc); disintegrates (e.g. starch, sodium starch glycolate, etc); or wetting agents (e.g. sodium lauryl sulphate, etc).

The compositions provided herein may additionally contain other adjunct components conventionally found in pharmaceutical compositions. Thus, for example, the compositions may contain additional compatible pharmaceutically-active materials or may contain additional materials useful in physically formulating various dosage forms of the composition of present invention, such as dyes, flavouring agents, preservatives, antioxidants, opacifiers, thickening agents and stabilizers. However, such materials, when added, should not unduly interfere with the biological activities of the components of the compositions provided herein.

Regardless of the method by which the constructs are introduced into a patient, colloidal dispersion systems may be used as delivery vehicles to enhance the in vivo stability of the construct and/or targeting the construct to a particular organ, tissue or cell type. Colloidal dispersion systems include, but are not limited to, macromolecule complexes, nanocapsules, microspheres, beads and lipid-based systems including oil-in-water emulsions, micelles, mixed micelles, liposomes and lipid:oligonucleotide complexes of uncharacterised structure. A preferred colloidal dispersion system is a plurality of liposomes. Liposomes are microscopic spheres having an aqueous core surrounded by one or more outer layers made up of lipids arranged in a bilayer configuration.

A therapeutically effective amount of construct is administered. The dose may be determined according to various parameters, especially according to the severity of the condition, age, and weight of the patient to be treated; the route of administration; and the required regimen. A physician will be able to determine the required route of administration and dosage for any particular patient. Optimum dosages may vary depending on the relative potency of individual constructs, and can generally be estimated based on EC50s found to be effective in vitro and in in vivo animal models. In general, dosage is from 0.01 mg/kg to 100 mg per kg of body weight. A typical daily dose is from about 0.1 to 50 mg per kg, preferably from about 0.1 mg/kg to 10 mg/kg of body weight, according to the potency of the specific construct, the age, weight and condition of the subject to be treated, the severity of the disease and the frequency and route of administration. Different dosages of the construct may be administered depending on whether administration is by intramuscular injection or systemic (intravenous or subcutaneous) injection. Preferably, the dose of a single intramuscular injection is in the range of about 5 to 20 ug. Preferably, the dose of single or multiple systemic injections is in the range of 10 to 100 mg/kg of body weight.

Due to construct clearance (and breakdown of any targeted molecule), the patient may have to be treated repeatedly, for example once or more daily, weekly, monthly or yearly. Persons of ordinary skill in the art can easily estimate repetition rates for dosing based on measured residence times and concentrations of the construct in bodily fluids or tissues. Following successful treatment, it may be desirable to have the patient undergo maintenance therapy, wherein the construct is administered in maintenance doses, ranging from 0.01 mg/kg to 100 mg per kg of body weight, once or more daily, to once every 20 years.

The invention is illustrated by the following Example:

### Example 1

Duchenne muscular dystrophy (DMD) is a severe muscle degenerative disorder characterized by mutations that disrupt the reading frame in the dystrophin (DMD) gene leading to the absence of functional protein (1). Antisense oligonucleotide (AO)— mediated exon skipping offers a potential therapy for DMD by restoring the open reading frame of mutant DMD transcripts (2-12), yielding the production of shorter functional forms of dystrophin protein that retain the critical amino terminal, cysteine rich and carboxy terminal domains necessary for function (13, 14). The therapeutic potential of this method has now been successfully shown in human subjects via local intramuscular AO injection (10).

To fully exploit AO-mediated splice correction as an effective therapy in DMD patients will require systemic correction of the DMD phenotype with increased potency. Systemic intravenous delivery of 2′-O-methyl phosphorothioate RNA and phosphorodiamidate morpholino oligomer (PMO) AOs have been shown to restore dystrophin expression in multiple peripheral muscles in mdx mice. However correction was of low efficiency for both AO types, and for the latter required a multiple dosing regimen comprising seven weekly doses of PMO at 100 mg/kg (3) to achieve a moderate restoration of dystrophin protein. Recently we and others have reported that PMO conjugated to short arginine-rich cell-penetrating peptides (CPPs) can induce effective systemic dystrophin exon skipping, including in cardiac muscle (15-18), showing the potential of PMO-peptide conjugates as therapeutic agents for DMD.

Few studies to date have investigated the possibility that cell-targeting peptides might permit enhanced in vivo tissue-specific nucleic acid delivery and activity. Although a recent report demonstrated successful transvascular nucleic acid delivery to brain using a neuronal targeting peptide derived from rabies virus glycoprotein complexed with double-stranded siRNA (19). We hypothesize such a cell-targeting approach may enhance AO delivery to muscle for DMD. In the present study we test this hypothesis by conjugating a muscle-specific heptapeptide peptide (MSP) (20) or a chimeric fusion peptide comprising MSP and a CPP (B peptide) to PMO, and evaluate these peptide-PMO conjugates in mdx mice. Our study shows for the first time that the chimeric peptide conjugate (B-MSP-PMO) induces highly effective systemic dystrophin exon skipping in mdx mice at doses as low as 6 mg/kg, with body-wide restoration of dystrophin protein and improvement of muscle pathology and function with no evidence of toxicity. This study demonstrates that such a chimeric peptide approach provides a safe and effective method for systemic AO delivery for DMD splice correction therapy and is likely to have broad utility.

### Materials and Methods

### Animals

6-8-week old mdx mice were used in all experiments (four mice each in the test and control groups). The experiments were carried out in the Animal unit, Department of Physiology, Anatomy and Genetics, University of Oxford, Oxford, UK according to procedures authorized by the UK Home Office. Mice were killed by CO2 inhalation or cervical dislocation at desired time points, and muscles and other tissues were snap-frozen in liquid nitrogen-cooled isopentane and stored at −80° C.

### PMO and PMO Peptide Conjugates

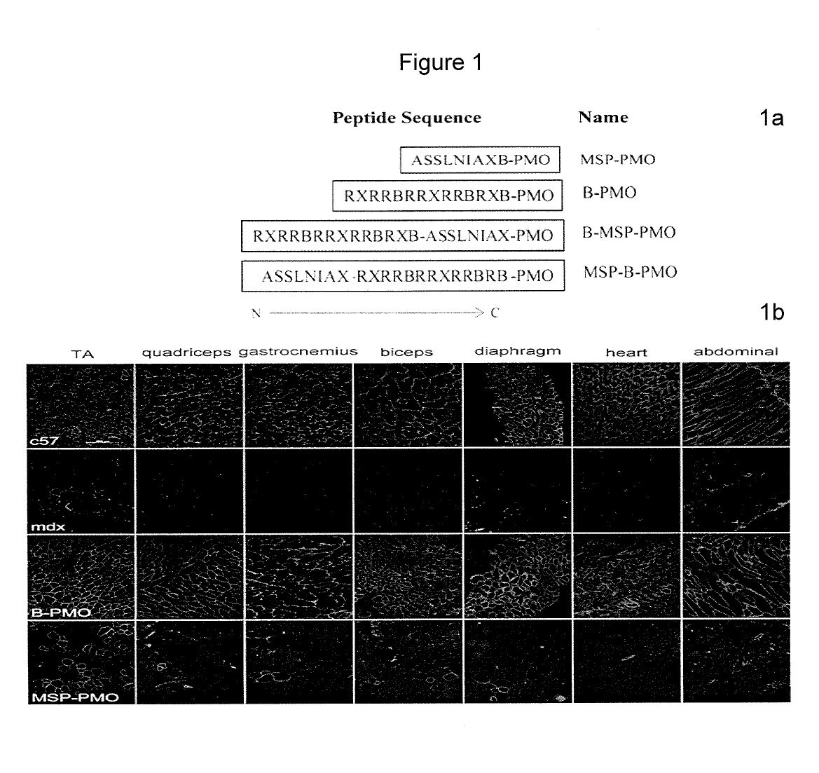

Four peptide-conjugated PMOs were synthesized and purified to >90% purity by AV1 Biopharma Inc. (Corvallis, Oreg., USA). The nomenclature and sequences of these constructs are shown in FIG. 1. The PMO AO was targeted to the murine dystrophin exon23/intron 23 boundary site. The four peptides are named as MSP, B, B-MSP, MSP-B. The PMO was conjugated to the carboxyl groups at the C-terminus of the four peptides using a method described elsewhere (27).

### Cell Culture and Transfection

The H₂K mdx myoblasts were cultured at 33° C. under a 10% CO₂/90% air atmosphere in high-glucose DMEM supplemented with 20% fetal calf serum, 0.5% chicken embryo extract (PAA laboratories Ltd, Yeovil, UK), and 20 units/ml γ-interferon (Roche applied science, Penzberg, Germany). Cells were then treated with trypsin and plated at 2×10⁴ cells per well in 24-well plates coated with 200 ug/ml gelatine (Sigma). H₂K mdx cells were transfected 24 h after trypsin treatment in a final volume of 0.5 ml of antibiotic- and serum-free Opti-MEM (Life Technologies). Each well was treated with 250 nM of PNA-peptide complexed with corresponding amounts of lipofectin (weight ratio 1:2=oligo:lipofectin) (Life Technologies) according to the supplier's instructions. After 4 h of incubation, the transfection medium was replaced with DMEM supplemented medium.

### RNA Extraction and Nested RT-PCR Analysis

Total RNA was extracted with Trizol (Invitrogen, UK) and 200 ng of RNA template was used for 20 μl RT-PCR with OneStep RT-PCR kit (Qiagen, UK). The primer sequences were used as previously reported (16). The products were examined by electrophoresis on a 2% agarose gel.

### Systemic Injections of Peptide-PMO Conjugates

Various amounts of PMO-peptide conjugates in 80 μl saline buffer were injected into tail vein of mdx mice at the final dose of 25 mg/kg, 30 mg/kg, 40 mg/kg, 3 mg/kg and 6 mg/kg, respectively. The animals were killed at various time points after injection by CO2 inhalation and tissues were removed and snap-frozen in liquid nitrogen-cooled isopentane and stored at −80° C.

Immunohistochemistry and Histology

Series of 8 μm sections were examined for dystrophin expression and dystrophin-associated protein complex (DAPC) with a series of polyclonal antibodies and monoclonal antibodies as described (16) Routine haematoxylin and eosin and Azan Mollary staining was used to examine overall muscle morphology and assess the level of infiltrating mononuclear cells and fibrosis.

### Centrally Nucleated Fibre Counts

TA, quadriceps and gastrocnemius muscles from mdx mice treated with PMO-peptide conjugates were examined. To ascertain the number of centrally nucleated muscle fibres, sections were stained for dystrophin with rabbit polyclonal antibody 2166 and counter-stained with DAPI for cell nuclei (Sigma, UK). About 500 dystrophin positive fibres for each tissue sample were counted and assessed for the presence of central nuclei using a Zeiss AxioVision fluorescence microscope. Fibres were judged centrally nucleated if one or more nuclei were not located at the periphery of the fibre. Untreated age-matched mdx mice were used as controls

### Protein Extraction and Western Blot

Protein extraction and Western blot were carried out as previously described (16). Various amounts protein from normal C57BL6 mice as a positive control and corresponding amounts of protein from muscles of treated or untreated mdx mice were used. The membrane was probed with DYS1 (monoclonal antibody against dystrophin R8 repeat, 1:200, NovoCastra, UK) for the detection of dytstrophin protein and aactinin as a loading control (mouse monoclonal antibody, 1:3000, Sigma, UK). The bound primary antibody was detected by horseradish peroxidise-conjugated goat anti-mouse IgGs and the ECL Western Blotting Analysis system (Amersham Pharmacia Biosciences, UK). The intensity of the bands obtained from treated mdx muscles was measured by Image J software; the quantification is based on band intensity and area, and is compared with that from normal muscles of C57BL6 mice.

### Functional Grip Strength Analysis

Treated mice and control mice were tested using a commercial grip strength monitor (Chatillon, UK). Each mouse was held 2 cm from the base of the tail, allowed to grip a protruding metal triangle bar attached to the apparatus with their forepaws, and pulled gently until they released their grip. The force exerted was recorded and 5 sequential tests were carried out for each mouse, averaged at 30 s apart.

### Clinical Biochemistry

Serum and plasma were taken from the mouse jugular vein immediately after the killing with CO2 inhalation. Analysis of serum creatinine kinase (CK), aspartate aminotransferase (AST), alanine aminotransferase (ALT), urea and creatinine levels was performed by the clinical pathology laboratory (Mary Lyon Centre, Medical Research Council, Harwell, Oxfordshire, UK).

### Tissues Biodistribution Analysis

Tissues were thawed at room temperature and then pre-weighed into individual 1.5 ml eppendorf tubes. Lysis buffer containing trypsin and proteinase K was added to pre-weighed tissue. Samples were placed into a shaking incubator temperature controlled at 60° C. overnight. After incubation, samples were centrifuged at 14000 g for 10 minutes and the supernatant was collected. Lysates were extracted 3:1 in acetonitrile, frozen on dry ice, and lyophilized. Lyophilized samples were reconstituted in HBS-P buffer (BIAcore, Piscataway, N.J.) and transferred to a 96 well plate. Plates were spun down (1000 g, 10 minutes) to pellet any particulate matter. Surface Performance Resonance (SPR) detection was performed on a Biacore T100 (GE/BIAcore, Piscataway, N.J.) instrument operating at 25° C. A CM dextran matrix pre-immobilized streptavidin sensor chip was bound with a biotin-labelled cDNA (Integrated DNA technologies) complementary to the PMO sequence. Target immobilization level for SA chip was set to maximum. Ligand was immobilized in a flow of 10 ul/min. The chip was fully saturated in a single 10 minute pulse and resulting in 1345 RU immobilized on the surface. The contact time during the concentration measurements was 120 seconds at a flow 30 ul/min followed by a dissociation time of 15 seconds. The DNA surface was regenerated with a single pulse (5 sec, 50 ul/min) of 10 mM glycine-HCl at pH 1.75. Biacore Concentration Analysis: A direct binding assay was used to determine tissue concentrations. Calibration was performed by spiking blank matrix with known concentrations of PMO at 100, 50, 25, 12.5, 6.25, 3.125, 0 nM. Three controls 1, 10, and 50 nM, were run every 15 cycles to assess integrity of calibration over time. Blank tissues were used to establish the limits of detection.

### Statistical Analysis

All data are reported as mean values ±SEM. Statistical differences between treatment groups and control groups were evaluated by SigmaStat (Systat Software, UK) and student's t test was applied.

### Results

MSP-PMO Conjugate is Much Less Effective than B-PMO for Dystrophin Splice Correction in Mdx Mice

To test the ability of cell-targeting peptides to enhance systemic dystrophin correction in mdx mice, we investigated a muscle-specific heptapeptide (MSP), previously identified by in vivo phage display as having increased muscle- and cardiac-binding properties (20), for its ability to enhance PMO splice correcting activity in muscle. We compared the MSP-PMO conjugate directly with the previously studied B-PMO conjugate (see FIG. 1a for the oligonucleotide and peptide sequences) in mdx mice at a 25 mg/kg single intravenous dose as B-PMO had been previously shown to restore expression of dystrophin in the tibialis anterior (TA) muscle by a single intramuscular injection (16). Three weeks following the single injection all skeletal muscle groups analysed demonstrated near normal levels of dystrophin protein by immunostaining following treatment with the B-PMO conjugate (FIG. 1b), consistent with previous reports (18). Surprisingly, the activity of MSP-PMO was found to be low, although more effective than PMO alone at the same dose (data not shown). High levels of dystrophin exon skipping and protein restoration were detected in hind limb, forelimb, abdominal wall and diaphragm muscles and also in cardiac tissue in mdx mice treated with the B-PMO conjugate, shown by RT PCR and Western blot. Increased levels of cardiac dystrophin restoration with the B-PMO conjugate were seen with higher intravenous doses of 30 and 40 mg/kg, which showed about 20% and 50% of normal levels respectively as indicated by Western blot. Moreover, BPMO also restored components of the dystrophin-associated protein complex (DAPC) (21, 22), which in the absence of functional dystrophin fail to localise accurately to the muscle sarcolemma. As a result, using a functional test of grip force strength (23,24), mdx mice treated with B-PMO were found to have significantly improved grip strength to within the normal range compared with untreated mdx mice.

### Chimeric B-MSP-PMO Induces Efficient Dystrophin Splice Correction

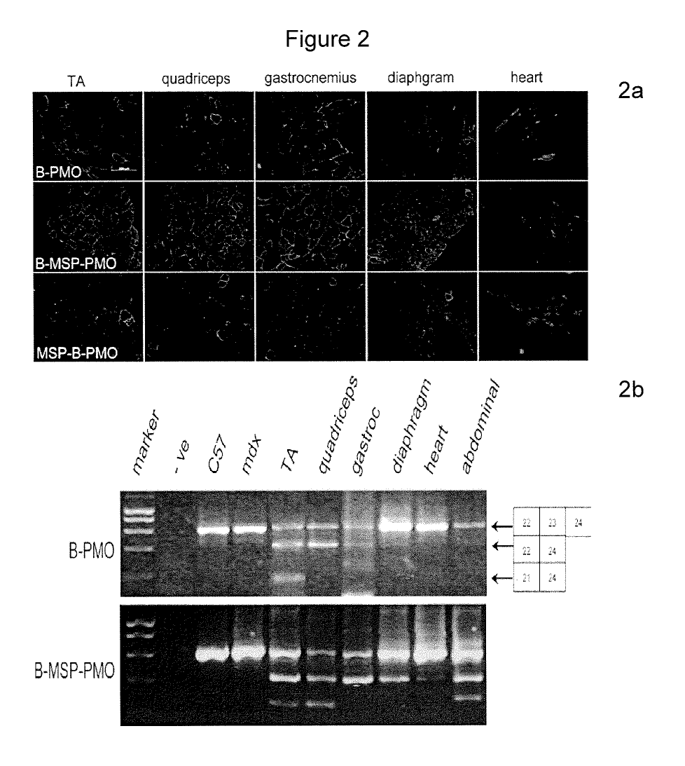

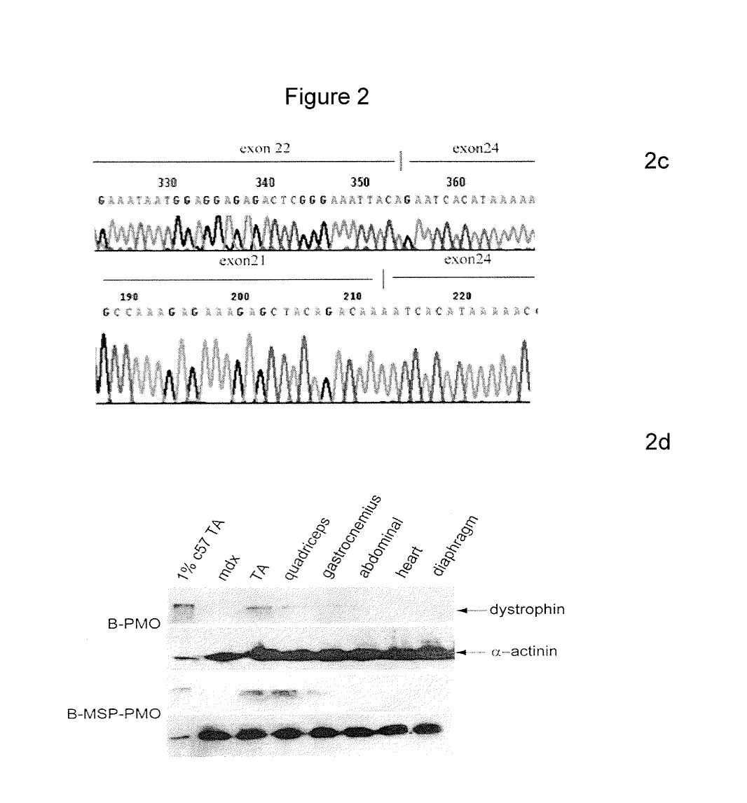

Since MSP has a high affinity for skeletal and cardiac muscle (20) we hypothesised that the poor activity of the MSP-PMO conjugate might be due to its weak ability to facilitate PMO internalisation following tissue localisation. We therefore tested whether fusion of the MSP motif to the B-peptide to generate a chimeric fusion peptide could improve its activity following systemic delivery. We tested two conjugated forms of this chimeric peptide, B-MSP-PMO, in which the MSP domain was positioned between the B and PMO sequences, and MSP-B-PMO in which the MSP domain was positioned away from PMO (FIG. 1a). In order to discover whether either of these conjugates provided enhanced activity over the B-PMO conjugate we investigated a low dose multiple injection protocol of 3 mg/kg in six weekly intravenous injections, reasoning that differences in efficacy would be most apparent at lower doses. Surprisingly, B-MSP-PMO, not MSP-B-PMO, proved highly effective in its ability to restore dystrophin expression in multiple skeletal muscle groups at this low dose compared with B-PMO. Widespread, uniform dystrophin expression was found throughout muscle cross-sections with the B-MSP-PMO conjugate, whereas fewer dystrophin-positive fibres were detected following B-PMO treatment at this dose. Virtually no dystrophin expression was detected with the alternative chimeric peptide PMO conjugate (MSP-B-PMO) (FIG. 2a).

No detectable dystrophin expression in heart was found with all three conjugates at this dose. The most striking difference between B-MSP-PMO and BPMO conjugates was seen in abdominal and diaphragm muscles; no detectable exon skipping products were found with B-PMO in these two tissues whereas approximately 20% of exon 23 transcripts were skipped with B-MSP-PMO as shown by RT-PCR (FIG. 2b) and confirmed by sequence analysis (FIG. 2c). It should be noted that RT-PCR is likely to overestimate the proportion of skipped transcripts given that full-length transcripts containing the nonsense mutation will be subject to nonsense-mediated decay. Western blot analysis showed that about 5% of the normal level of dystrophin was restored in TA and quadriceps muscles with B-MSP-PMO, whereas only ˜1% was observed in the same tissues with B-PMO (FIG. 2d). Consistent with the immunostaining data, minimal exon skipping activity and protein restoration were found with the MSP-B-PMO conjugate (data not shown).

Enhanced Systemic Exon Skipping Efficiency with B-MSP-PMO in Body-Wide Skeletal Muscles

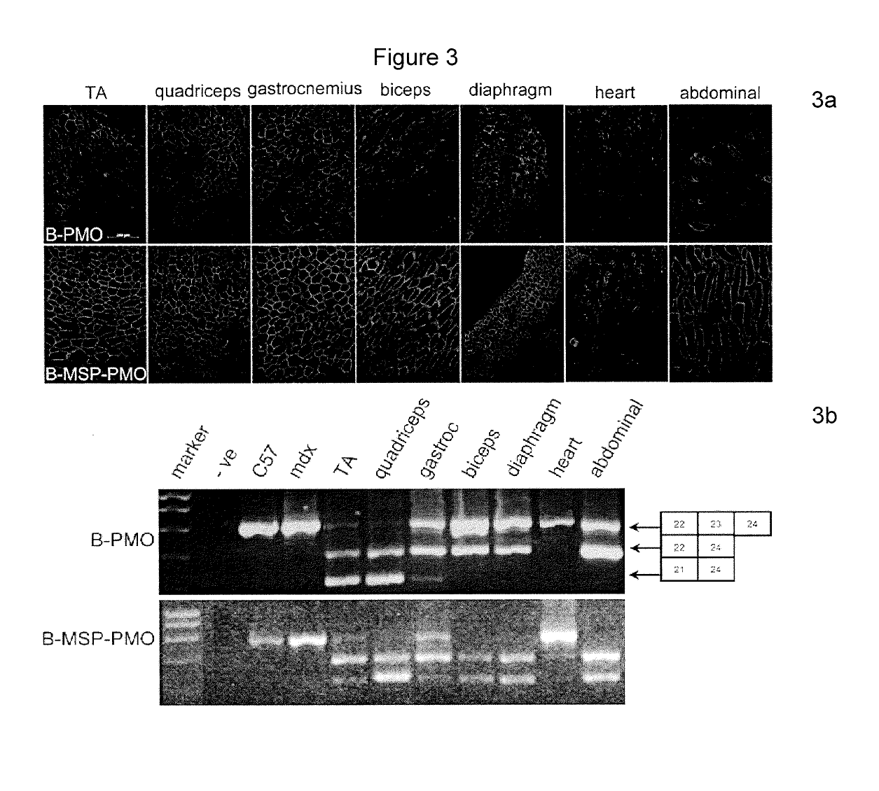

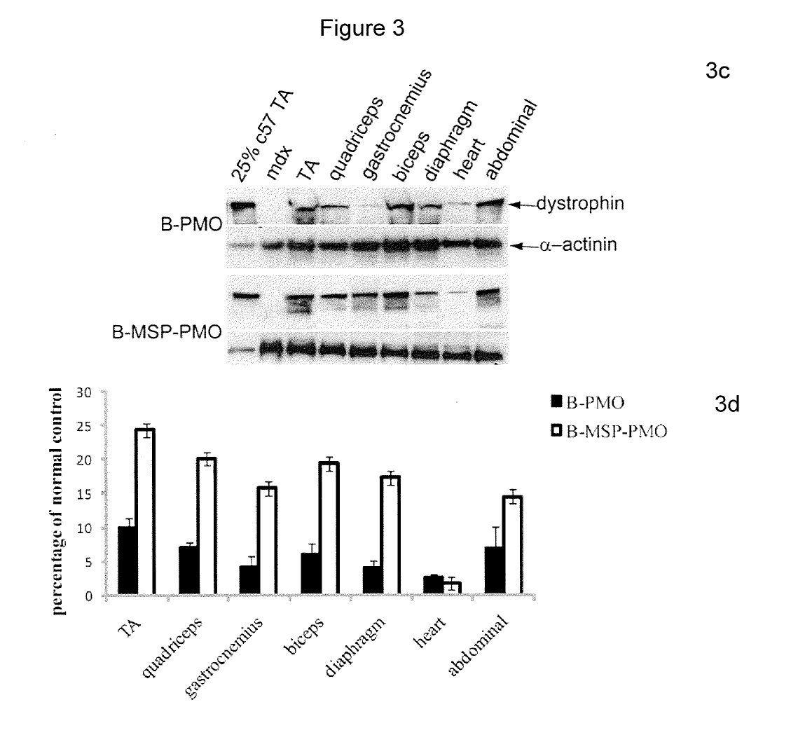

To fully explore the splice-correcting potential of the B-MSP-PMO conjugate harbouring both muscle-targeting heptapeptide and arginine-rich CPP domains, we optimised the dosing regimen by administering the same total dose of 1 8 mg/kg over three weekly intravenous injections of 6 mg/kg each. When compared directly with BPMO, B-MSP-PMO proved highly efficacious at this dose giving high-level body-wide correction of dystrophin protein expression in multiple peripheral skeletal muscles, although only at low levels in heart (FIG. 3a). Little variation in dystrophin exon skipping efficiency was observed between different muscle groups treated with B-MSP-PMO as has been reported previously following naked PMO treatment (3). Enhanced exon skipping efficiency of the B-MSP-PMO conjugate was seen by RT-PCR, with negligible full-length uncorrected dystrophin transcripts detectable in biceps, abdominal and diaphragm muscles (FIG. 3b). Up to 25% of the normal level of dystrophin protein was restored in skeletal muscles of mdx mice treated with B-MSP-PMO compared with the B-PMO conjugate, which showed approximately 10% of normal levels as indicated by Western blot (FIGS. 3c and 3d). These results clearly demonstrated that the B-MSP-PMO conjugate facilitated enhanced dystrophin splice correction compared with B-PMO lacking the MSP domain.

Functional and Phenotypic Improvement of the Mdx Mouse with B-MSP-PMO Treatment

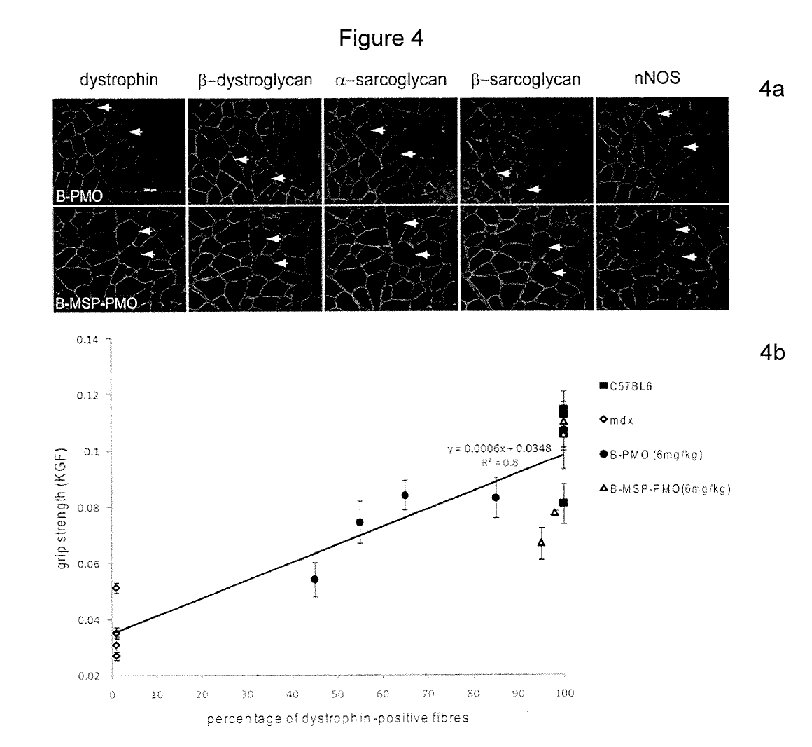

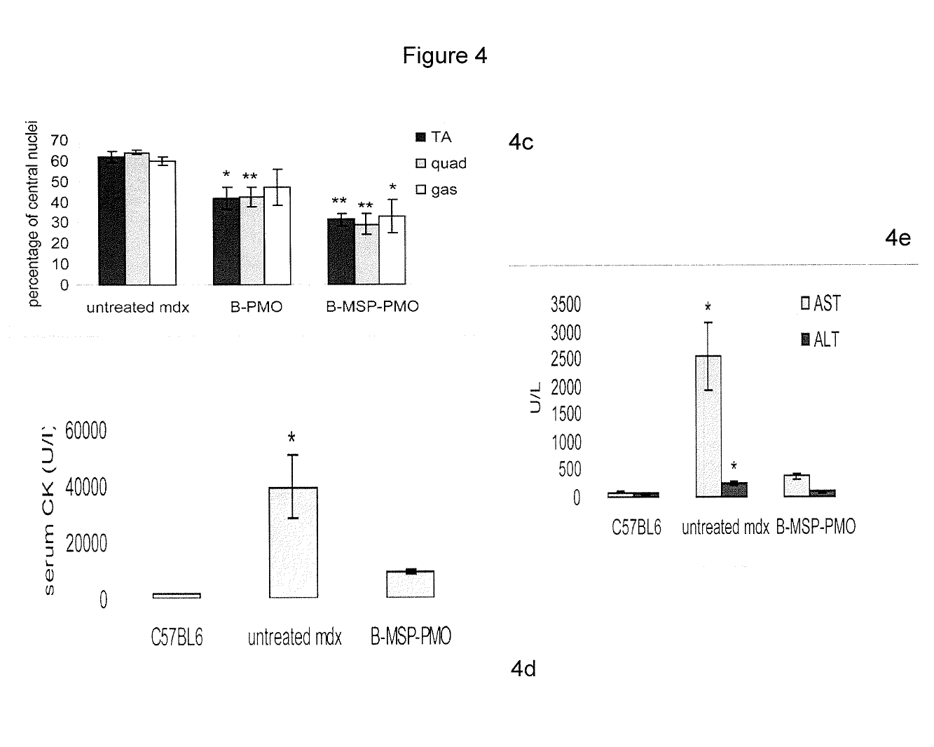

Given the high activity of the B-MSP-PMO conjugate, we next examined its ability to restore function and correct disease pathology in mdx mice. First we evaluated DAPC expression in mdx mice treated with the 6 mg/kg dose regimen. Serial immunostaining showed restored expression and correct localisation of DAPC component proteins Pdystroglycan, α-sarcoglycan and β-sarcoglycan in B-MSP-PMO and B-PMO treated mdx mouse TA muscles compared with untreated mdx mice (FIG. 4a). The DAPC also has important signalling functions via nNOS (21) and its restoration and correct localisation was also detected following B-MSP-PMO treatment (FIG. 4a). Physically functional improvement was measured using grip strength tests, which test predominantly but not exclusively forelimb functional restoration (23,24). B-MSPPMO treated animals showed significant strength improvement to within the normal range compared with untreated age-matched mdx controls, indicating a degree of functional recovery and close correlation with the percentage of dystrophin-positive fibres in treated biceps (FIG. 4b). Routine H&E and Azan Mollary histology of BMSP-PMO treated muscles showed no overt evidence of toxicity and fibrosis and analysis of the number of centrally nucleated myofibres, an index of ongoing degeneration/regeneration cycles (25,26), revealed a significantly decreased level of degeneration and regeneration in TA, quadriceps and gastrocnemius muscles in mdx mice treated with the B-MSP-PMO conjugate (p<0.001) compared with untreated age-matched control mice (FIG. 4c). Finally, we analysed serum biochemistry indices including creatinine kinase (CK), an index of ongoing muscle injury (25). This demonstrated significantly lower CK levels following B-MSP-PMO treatment than in untreated control mice (FIG. 4d), demonstrating the protective effects of systemic dystrophin restoration on myofibre integrity. Serum biochemistry including aspartate aminotransferase (AST) and alanine aminotransferase (ALT) enzyme levels as indices of liver function also showed significant decreases compared with untreated controls and fell to within the normal range in B-MSP-PMO treated animals (FIG. 4e). No change was observed in the levels of urea and creatinine in the B-MSP-PMO treated mdx mice, indicating no obvious renal toxicity (data not shown).

### DISCUSSION

Here we demonstrate for the first time that a PMO oligomer conjugated to a chimeric fusion peptide (B-MSP-PMO) comprising a muscle-targeting domain and an arginine-rich cell penetrating peptide domain, directs highly effective dystrophin protein restoration, muscle function restoration and correction of the dystrophic phenotype in mdx mice. Our data shows that the B-MSP-PMO conjugate has significant potential for enhanced restoration of dystrophin expression and arresting DMD pathology at very low systemic doses, compatible with successful application in human subjects. A previous study reported use of a fusion peptide comprising cell-targeting and arginine-rich peptide domains for siRNA delivery to brain (19). The present study is the first to show such chimeric peptide approach to AOs can permit enhanced systemic correction of a genetic defect in an animal model of human disease.

We and others have recently reported that short arginine-rich CPPs directly conjugated to PMO can induce efficient systemic splice correction in mdx mice (15-18), providing a significant advance on previous studies using systemic naked AO delivery for DMD (3,8). In the present study the hypothesis that PMO conjugation to a cell-targeting peptide domain can induce enhanced muscle delivery and further improve the efficacy of systemic DMD splice correction has been tested. The MSPPMO conjugate proved surprisingly ineffective. A possible explanation for this is that this cell-targeting peptide alone may direct the AO conjugate to the targeted cells in the absence of efficient internalization. Further studies will be needed to understand the delivery pathway and mechanism. However, the chimeric peptide with the B-MSP combination proved highly effective in inducing dystrophin splice correction and restoring the expression of dystrophin protein in body-wide skeletal muscles compared with the conjugate lacking the MSP domain. Utilising very low B-MSP-PMO doses of 6 mg/kg in mdx mice, we have now shown highly efficient correction of dystrophin protein in multiple skeletal muscles (FIGS. 3c and 3d), restoration of DAPC structural integrity (FIG. 4a), significant improvement in muscle strength which correlated closely with the percentage of dystrophin-positive fibres (FIG. 4b) and correction of the mdx dystrophic phenotype (FIGS. 4c-e). The superior activity of the B-MSP-PMO conjugate to B-PMO alone in enhancing systemic dystrophin splice correction in mdx mice was also shown in a lower dose study (3 mg/kg dose). Overall these findings indicate that the MSP cell-targeting peptide fails to augment systemic splice correction in the absence of an arginine-rich transduction domain, but that when coupled together in a chimeric fusion peptide the MSP peptide significantly enhances systemic PMO activity.

Surprisingly, the chimeric peptide with the MSP-B combination showed little activity in restoring the expression of dystrophin (FIG. 2a). Subsequent studies with fluorescein-labelled PMO AO conjugates both in vitro and in vivo have shown that internalization of PMO was facilitated by the B-MSP fusion peptide whereas the alternative MSP-B peptide failed to provide efficient cell uptake (Yin et al., in preparation). Therefore, although the mechanism is unclear, the location of an MSP domain within the chimeric fusion peptide is position-dependent in order to facilitate the effective internalization of AO-peptide conjugates.

In order to verify the cell-targeting role of MSP, we quantified the PMO concentration in muscles from the mdx mice treated with B-MSP-PMO and B-PMO at the 6 mg/kg dose. The tissue distribution data demonstrated higher tissue uptake for B-MSP-PMO compared with B-PMO in most muscle groups although the difference in uptake was not statistically significant except for the diaphragm. No significant differences were observed in non-muscle tissues such as liver and kidney between these two constructs (data not shown). Our hypothesis therefore is that the role of the fusion peptide is to allow greater internalization of AO into muscle cells. This is supported by in vitro data showing that B-MSP-PMO had the greatest efficacy in inducing exon skipping in mdx primary muscle cells compared with B-PMO and MSP-B-PMO over a range of concentrations (Wang et al., submitted).

That little evidence for correction of cardiac dystrophin expression was found (for BPMO as well as the B-MSP-PMO conjugate), is most likely due to the low doses utilised in this study and the 2-3 fold lower binding affinity that the MSP peptide has for cardiac compared with skeletal muscle (20). This is supported by the finding that approximately 15-20% of normal dystrophin protein was detected in heart when a single 25 mg/kg dose B-MSP-PMO was administered to mdx mice intravenously as compared with 10% for B-PMO (data not shown). Nevertheless, cardiac dystrophin correction by peptide-PMOs (B-MSP-PMO as well as B-PMO), even at higher doses, is clearly less efficient than that seen in peripheral muscles. While it is possible that exon skipping of the DMD pre-mRNA is less efficient in heart, efficient dystrophin correction is seen in primary cardiomyocytes in culture (Wang and Yin, submitted), and therefore the most likely explanation at present is that differences in the cardiac microvasculature and endothelial barrier prevent less efficient PMO access than occurs in peripheral muscle groups. Given the significant potential of the B-MSPPMO conjugate, detailed toxicological analysis and long-term studies will now determine whether it is suitable for clinical evaluation in DMD patients. Further studies of the B-MSP chimeric peptide, including investigation of the lack of efficacy of the MSP-B-PMO conjugate, will yield improved versions of this fusion peptide likely to have broad experimental and clinical utility.

### Example 2

We have previously demonstrated that the efficacy of an exon skipping PMO conjugated to a chimeric peptide consisting of a cell-penetrating peptide (B) and a muscle-targeting peptide (MSP) is dependent upon the orientation of these peptides with respect to PMO, with B-MSP-PMO being significantly more effective than MSP-B-PMO. To investigate the general significance of this observation, we replaced MSP with another muscle-targeting peptide, peptide 9 (or HSP), identified through an in vivo phage display screen and shown to have strong binding affinity to muscle and heart tissues. The sequence of peptide 9 (HSP) is shown in SEQ ID NO: 46.

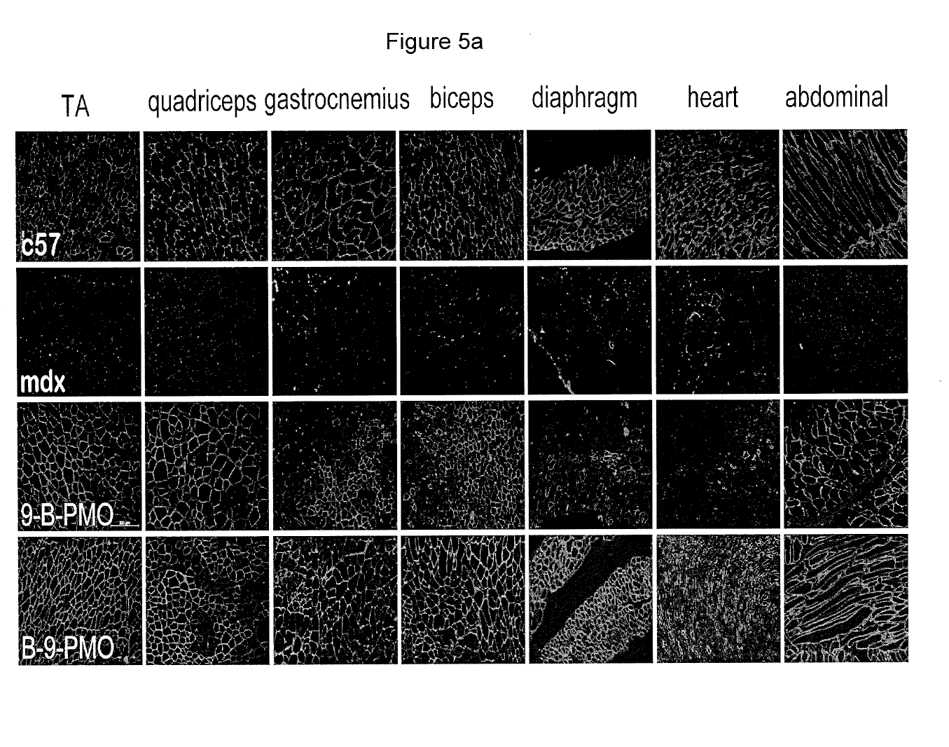

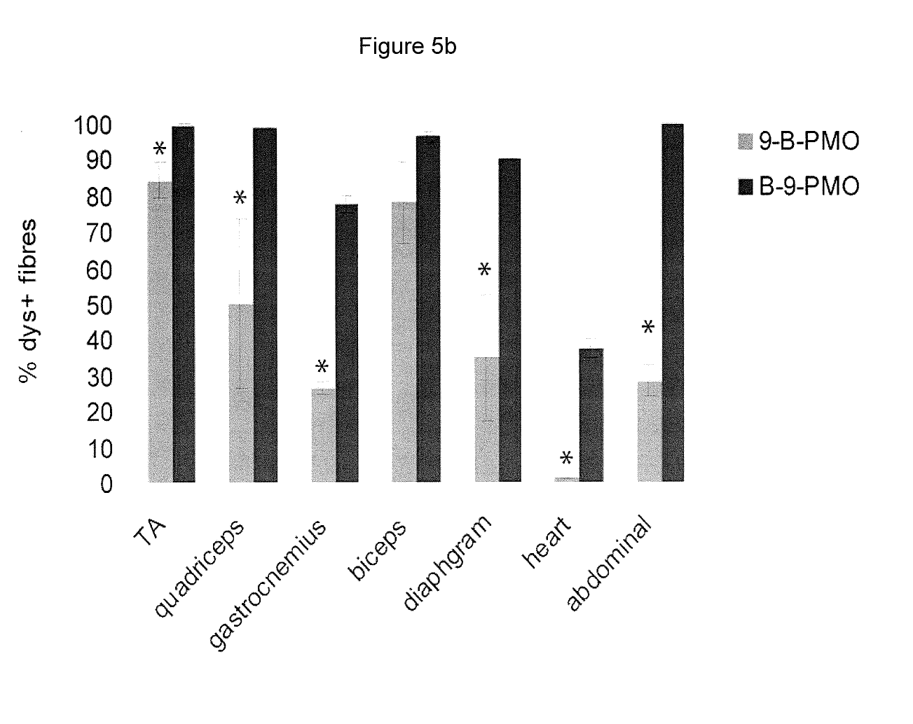

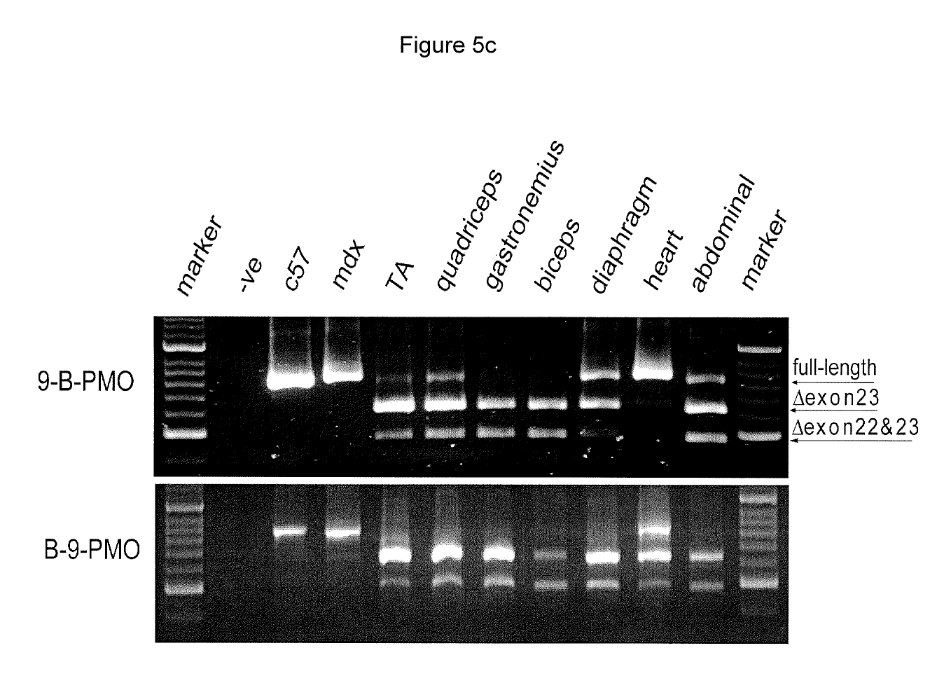

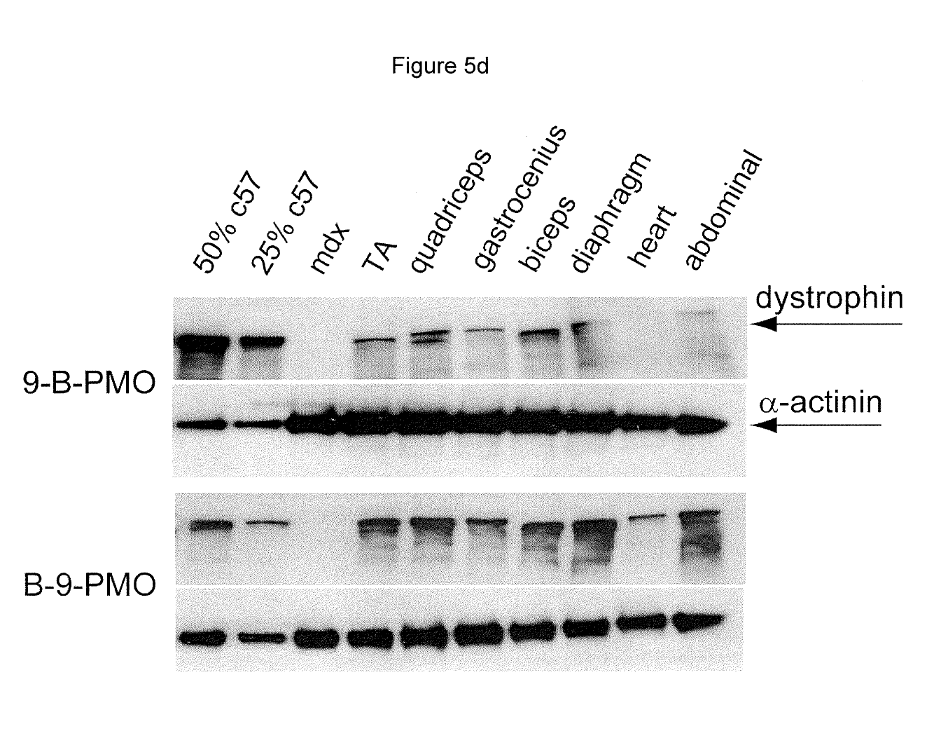

Body-wide muscles including the heart were evaluated for the efficiency of exon-skipping following a single intravenous injection of either B-9-PMO or 9-B-PMO in adult mdx mice at 25 mg/kg doses. Approximately 100% dystrophin-positive fibres were detected in tibialis anterior (TA), quadriceps, biceps and abdominal muscle cross-sections with B-9-PMO treatment as shown by immunohistochemical staining, whereas a significantly lower level of dystrophin expression was observed in the corresponding muscles treated with 9-B-PMO (FIGS. 5a and 5b). No detectable unskipped dystrophin transcript was observed in any peripheral muscles treated with B-9-PMO and even in heart, greater than 50% exon skipping was detected at the RNA level (FIG. 5c). Up to 65% of total dystrophin protein was restored in all the peripheral muscles treated with B-9-PMO and about 25% of normal levels of dystrophin protein restored in heart as determined by Western blot (FIG. 5d). In contrast, 9-B-PMO demonstrated a significantly reduced activity in all muscles in comparable assays.

This result is consistent with our previous report identifying B-MSP-PMO and supports the hypothesis that the activity of chimeric peptide-PMO conjugates is dependent on alignment of the tissue-specific peptide with respect to the arginine-rich domain and the PMO sequence, with B-MSP-PMO and now B-9-PMO having significantly enhanced activity compared with the reverse order chimeric peptide-PMO conjugates.

### Example 3

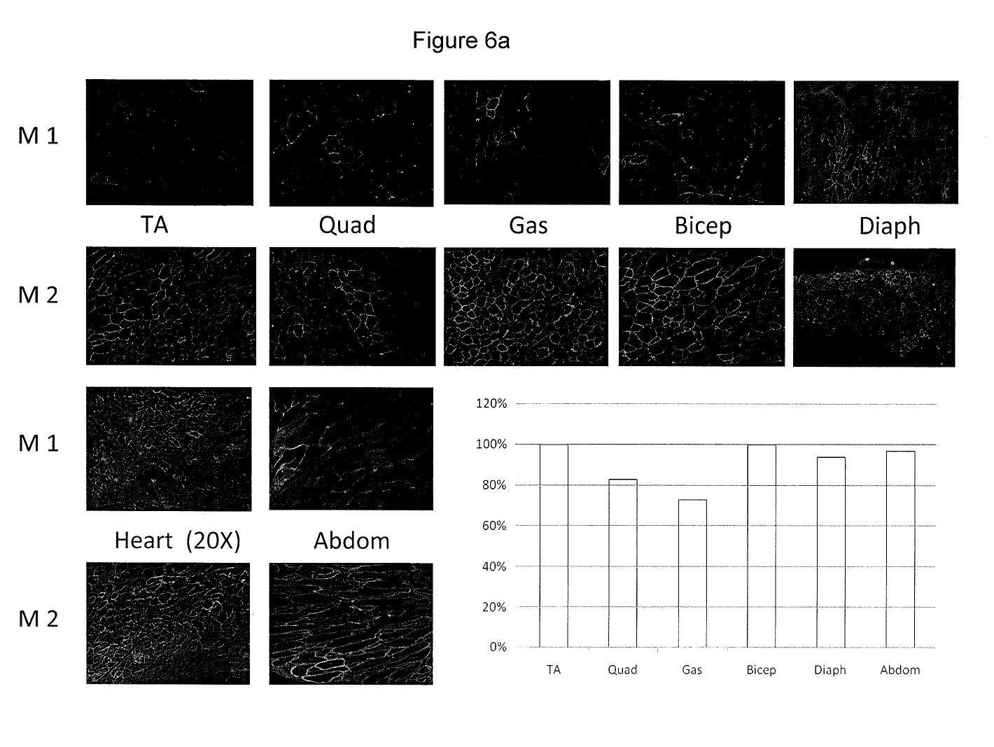

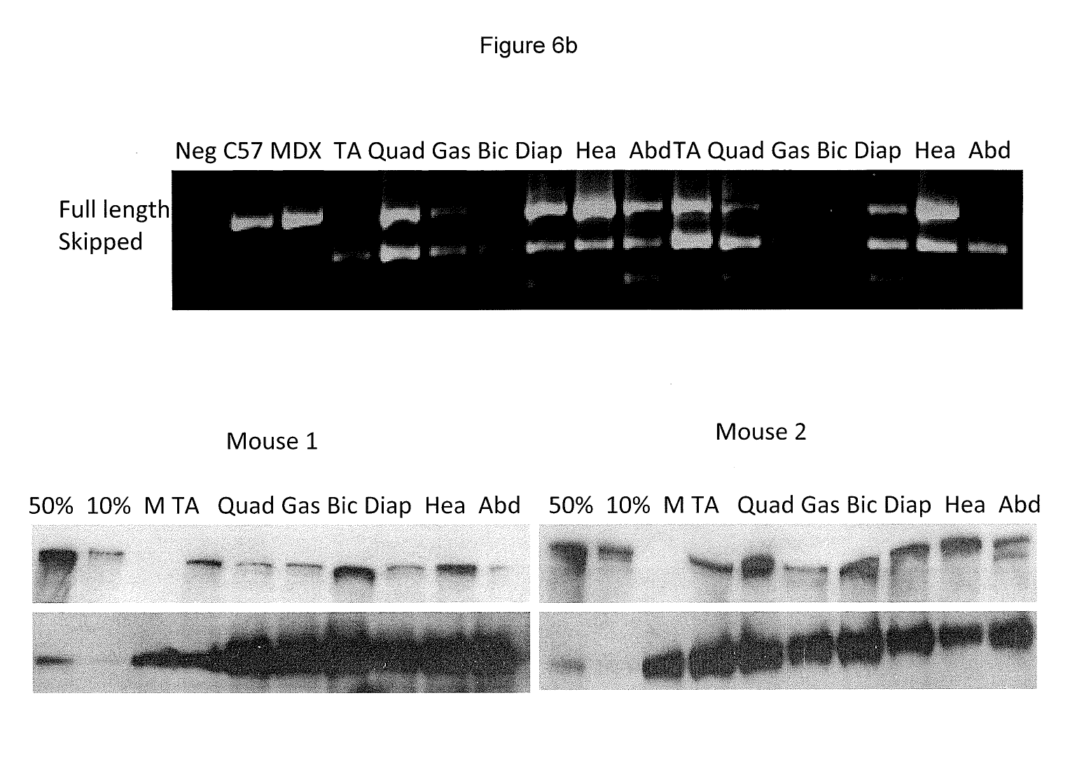

We have also demonstrated the efficacy of an exon skipping PMO conjugated to a chimeric peptide consisting of Pip5e and MSP (SEQ ID NO: 51). The results are shown in FIGS. 6a and 6b.

### Example 4

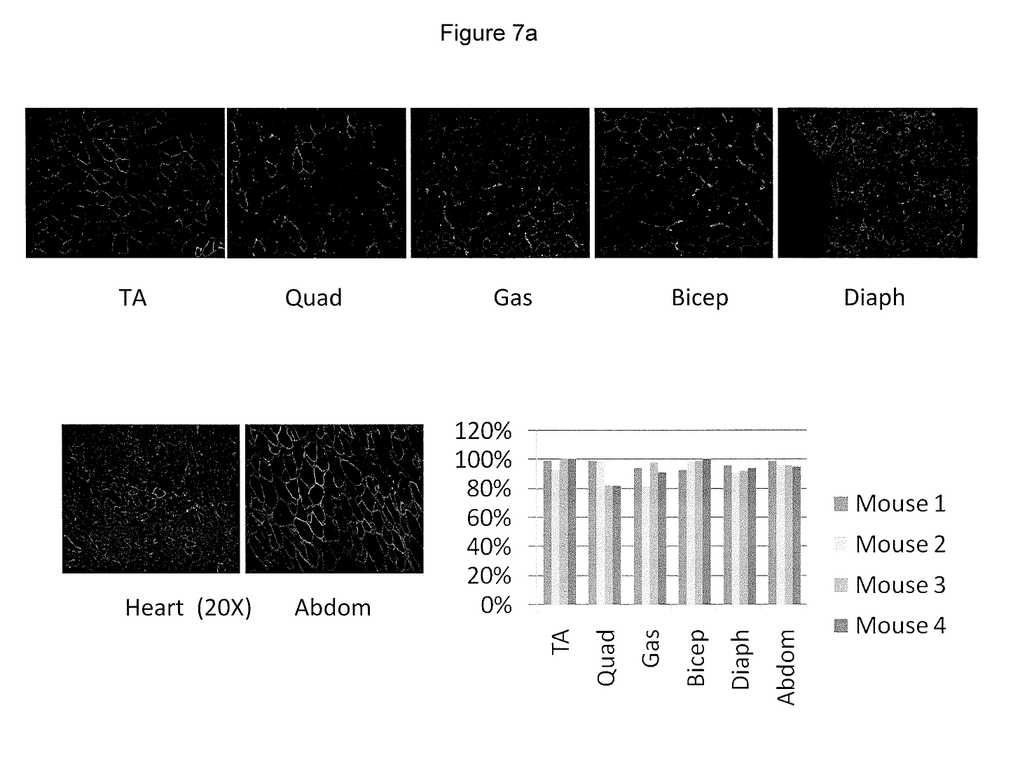

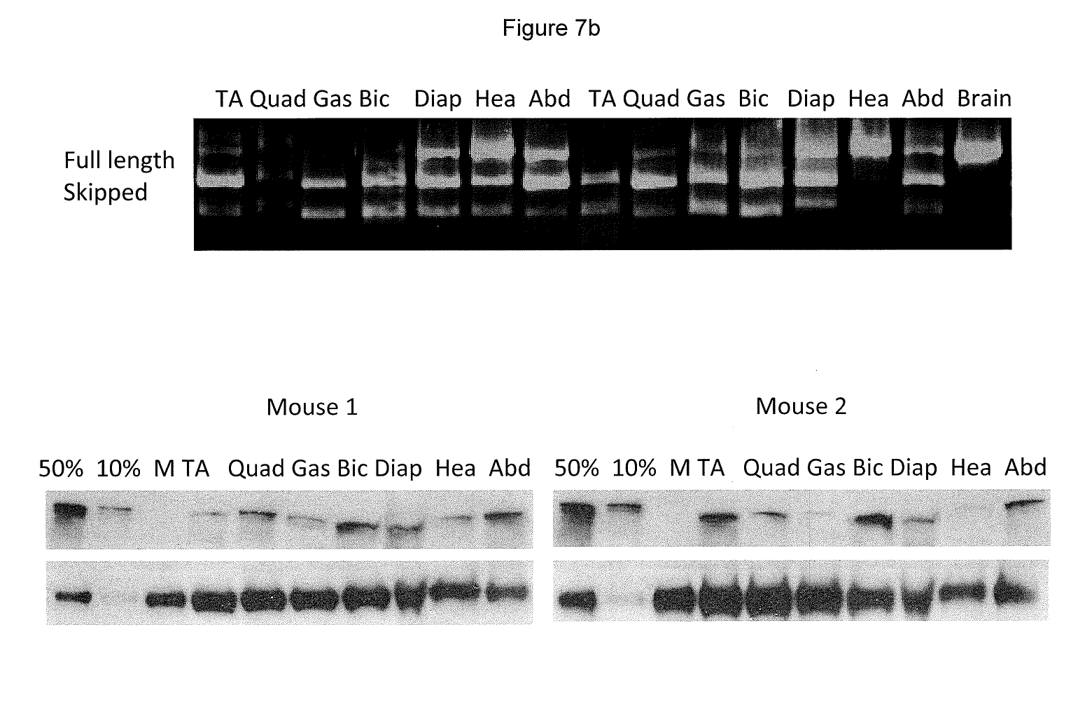

We have also demonstrated the efficacy of an exon skipping PMO conjugated to the chimeric peptide RXB-MSP-RXB (SEQ ID NO: 52). The results are shown in FIGS. 7a and 7b.

### REFERENCES

1. Hoffman, E. P., Brown, R. H., and Kunkel, L. M. (1987) Dystrophin: the protein product of the Duchenne muscular dystrophy locus. Cell, 51, 9 19-928.
2. Aartsma-Rus, A., Kaman, W. E., Weij, R., den Dunnen, J. T., van Ommen, G. J., and van Deutekom, J. C. (2006) Exploring the frontiers of therapeutic exon skipping for Duchenne muscular dystrophy by double targeting within one or multiple exons. Mol. Ther., 14, 401-407.
3. Alter, J., Lou, F., Rabinowitz, A., Yin, H., Rosenfeld, J., Wilton, S. D., Partridge, T. A., and Lu, Q. L. (2006) Systemic delivery of morpholino oligonucleotide restores dystrophin expression bodywide and improves dystrophic pathology. Nat. Med., 12, 175-177.
4. Gebski, B. L., Mann, C. J., Fletcher, S., and Wilton, S. D. (2003) Morpholino antisense oligonucleotide induced dystrophin exon 23 skipping in mdx mouse muscle. Hum. Mol. Genet., 12, 1801-1811.
5. Aartsma-Rus, A., Janson, A. A., Kaman, W. E., Bremmer-Bout, M., den Dunnen, J. T., Baas, F., van Ommen, G. J., and van Deutekom, J. C. (2003) Therapeutic antisense-induced exon skipping in cultured muscle cells from six different DMD patients. Hum. Mol. Genet., 12, 907-914.
6. Aartsma-Rus, A., Janson, A. A., Kaman, W. E., Bremmer-Bout, M., van Ommen, G. J., den Dunnen, J. T., and van Deutekom, J. C. (2004) Antisense-induced multiexon skipping for Duchenne muscular dystrophy makes more sense. Am. J. Hum. Genet., 74, 83-92.
7. Lu, Q. L., Mann, C. J., Lou, F., Bou-Gharios, G., Morris, G. E., Xue, S. A., Fletcher, S.,
Partridge, T. A., and Wilton, S. D. (2003), Functional amounts of dystrophin produced by skipping the mutated exon in the mdx dystrophic mouse. Nat. Med., 9, 1009-1014.
8. Lu, Q. L., Rabinowitz, A., Chen, Y. C., Yokota, T., Yin, H., Alter, J., Jadoon, A., Bou-Gharios, G., and Partridge, T. (2005) Systemic delivery of antisense oligoribonucleotide restores dystrophin expression in body-wide skeletal muscles. Proc. Natl. Acad. Sci. USA, 102, 198-203.
9. Mann, C. J., Honeyman, K., Cheng, A. J., Ly, T., Lloyd, F., Fletcher, S., Morgan, J. E., Partridge, T. A., and Wilton, S. D. (2001) Antisense-induced exon skipping and synthesis of dystrophin in the mdx mouse. Proc. Natl. Acad. Sci. USA, 98, 42-47.
10. van Deutekom, J. C., Janson, A. A., Ginjaar, I. B., Frankhuizen, W. S., Aartsma-Rus, A., Bremmer-Bout, M., den Dunnen, J. T., Koop, K., van der Kooi, A. J., Goemans, N. M. et al. (2007) Local dystrophin restoration with antisense oligonucleotide PRO051. N. Engl. J. Med., 357, 2677-2686.
11. Wilton, S. D., Fall, A. M., Harding, P. L., McClorey, G., Coleman, C., and Fletcher, S. (2007) Antisense oligonucleotide-induced exon skipping across the human dystrophin gene transcript. Mol. Ther., 15, 1288-1296.
12. Yin, H., Lu, Q., and Wood, M. (2008) Effective exon skipping and restoration of dystrophin expression by peptide nucleic acid antisense oligonucleotides in mdx mice. Mol. Ther., 16, 38-45.
13. England, S. B., Nicholson, L. V., Johnson, M. A., Forrest, S. M., Love, D. R., Zubrzycka-Gaarn, E. E., Bulman, D. E., Harris, J. B., and Davies, K. E. (1990) Very mild muscular dystrophy associated with the deletion of 46% of dystrophin. Nature, 343, 180-182.
14. Gregorevic, P., Blankinship, M. J., Allen, J. M., Crawford, R. W., Meuse, L., Miller D. G., Russell, D. W., and Chamberlain, J. S. (2004) Systemic delivery of genes to striated muscles using adeno-associated viral vectors. Nat. Med., 10, 828-834.
15. Jearawiriyapaisarn, N., Moulton, H. M., Buckley, B., Roberts, J., Sazani, P., Fucharoen, S., Iversen, P. L., and Kole, R. (2008) Sustained Dystrophin Expression Induced by Peptide-conjugated Morpholino Oligomers in the Muscles of mdx Mice. Mol. Ther., 16(9), 1624-9.
16. Yin, H., Moulton, H. M., Seow, Y., Boyd, C., Boutilier, J., Iverson, P., and Wood. M. (2008) Cell-penetrating peptide-conjugated antisense oligonucleotides restore systemic muscle and cardiac dystrophin expression and function. Hum. Mol. Genet., 17, 3909-3918.
17. Wu, B., Li, Y., Morcos, P. A., Doran, T. J., Lu, P., and Lu, Q. L. (2009) Octaguanidine morpholino restores dystrophin expression in cardiac and skeletal muscles and ameliorates pathology in dystrophic mdx mice. Mol. Ther., 17, 864-871.
18. Wu, B., Moulton, H. M., Iversen, P. L., Jiang, J., Li, J., Li, J., Spurney, C. F., Sali, A., Guerron, A. D., Nagaraju, K., Doran, T., Lu, P., Xiao, X., Lu, Q. L. (2008) Effective rescue of dystrophin improves cardiac function in dystrophin-deficient mice by a modified morpholino oligomer. Proc. Natl. Acad. Sci. USA 105: 14814-14819.
19. Kumar, P., Wu, H., McBride, J. L., Jung, K. E., Kim, M. H., Davidson, B. L., Lee, S. K., Shankar, P., and Manjunath, N. (2007) Transvascular delivery of small interfering RNA to the central nervous system. Nature, 448, 39-43.
20. Samoylova, T. I., and Smith, B. F. (1999). Elucidation of muscle-binding peptides by phage display screening. Muscle Nerve, 22, 460-466.
21. Blake, D. J., Weir, A., Newey, S. E., and Davies, K. E. (2002) Function and genetics of dystrophin and dystrophin-related proteins in muscle. Physiol. Rev., 82, 291-329.
22. Tinsley, J. M., Blake, D. J., Zuellig, R. A., and Davies, K. E. (1994) Increasing complexity of the dystrophin-associated protein complex. Proc. Natl. Acad. Sci. USA, 91, 8308-8313.
23. Fowler, S. C., Zarcone, T. J., Chen, R., Taylor, M. D., and Wright, D. E. (2002) Low grip strength, impaired tongue force and hyperactivity induced by overexpression of neurotrophin-3 in mouse skeletal muscle. Int. J. Dev. Neurosci., 20, 303-308.
24. Qiao, C., Li, J., Jiang, J., Zhu, X., Wang, B., Li, J., and Xiao, X. (2008) Myostatin propeptide gene delivery by adeno-associated virus serotype 8 vectors enhances muscle growth and ameliorates dystrophic phenotypes in mdx mice. Hum. Gene. Ther., 19, 241-254.
25. Glesby, M. J., Rosenmann, E., Nylen, E. G., and Wrogemann, K. (1988) Serum CK, calcium, magnesium, and oxidative phosphorylation in mdx mouse muscular dystrophy. Muscle Nerve, 11, 852-856Wells, D. J., Wells, K. E., Walsh, F. S., Davies, K. E., Goldspink, G., Love, D. R., ChanThomas, P., Dunckley, M. G., Piper, T., and Dickson, G. (1992) Human dystrophin expression corrects the myopathic phenotype in transgenic mdx mice. Hum. Mol. Genet., 1, 35-40.
26. Wu, R. P., Youngblood, D. S., Hassinger, J. N., Lovejoy, C. E., Nelson, M. H., Iversen, P. L., and Moulton, H. M. (2007) Cell-penetrating peptides as transporters for morpholino oligomers: effects of amino acid composition on intracellular delivery and cytotoxicity. Nucleic. Acid. Res., 35(15), 5182-5 191.

1. A construct suitable for delivery of a biologically active compound into cells, comprising:
(a) a positively charged peptide;
(b) a targeting-delivery peptide; and
(c) the biologically active compound;
wherein the positively charged peptide is covalently attached to the targeting-delivery peptide and the biologically active compound is covalently or non-covalently attached to the resultant chimeric cell delivery peptide.

2. A construct according to claim 1 wherein the positively charged peptide is covalently attached to the amino terminus of the targeting-delivery peptide.

3. A construct according to claim 1 wherein the positively charged peptide is arginine rich.

4. A construct according to claim 1 wherein at least 20% of the amino acids in the positively charged peptide are arginine (R).

5. A construct according to claim 1 wherein the positively charged peptide comprises a sequence of the formula (RZR(Z)ₗ(ILFQY)ₘ)ₙ or a functional derivative thereof, wherein Z is an aminoalkyl spacer, l is 0 or 1, m is 0 or 1 and n is from 2 to 6.

6. A construct according to claim 5 wherein Z is 6-aminohexanoyl (X) or betaalanyl (B).

7. A construct according to claim 5 wherein the positively charged peptide comprises any of the sequences selected from the group consisting of: SEQ ID NOS:1-44, RXRZRXR, RBRZRBR, RXRZRBR, RBRZRXR, RXRZRXRRXR(RXRZ-SEQ ID NO: 1), RXRRXRZRXR (SEQ ID NO: 1-ZRXR), RXRZRXRZRXR, RXRILFQYRXRZRXR (SEQ ID NO: 5-ZRXR), RXRZRXRILFQYRXR(RXRZ-SEQ ID NO: 5), RBRZRBRRBR(RBRZ-SEQ ID NO: 2), RBRRBRZRBR (SEQ ID NO: 2-ZRBR), RBRYRBRZRBR (SEQ ID NO: 22-ZRBR), RBRZRBRILFQYRBR(RBRZ-SEQ ID NO: 6), RBRILFQYRBRZRBR (SEQ ID NO: 6-ZRBR), RXRZRBRRXR(RXRZ-SEQ ID NO: 4), RXRRBRZRXR (SEQ ID NO: 4-ZRXR), RXRZRBRZRXR, RXRZRBRILFQYRXR(RXRZ-SEQ ID NO: 8), RXRILFQYRBRZRXR (SEQ ID NO: 7-ZRXR), RXRZRBRRBR(RXRZ-SEQ ID NO: 2), RXRRBRZRBR (SEQ ID NO: 3-ZRBR), RXRZRBRZRBR, RXRZRBRILFQYRBR(RXRZ-SEQ ID NO: 6), RXRILFQYRBRZRBR (SEQ ID NO: 7-ZRBR), RXRZRXRRBR(RXRZ-SEQ ID NO: 3), RXRRXRZRBR (SEQ ID NO: 1-ZRBR), RXRZRXRZRBR, RXRZRXRILFQYRBR(RXRZ-SEQ ID NO: 7), RXRILFQYRXRZRBR (SEQ ID NO: 5-ZRBR), RBRZRXRRBR(RBRZ-SEQ ID NO: 3), RBRRXRZRBR (SEQ ID NO: 4-ZRBR), RBRZRXRZRBR, RBRZRXRILFQYRBR (RBRZ-SEQ ID NO: 7), RBRILFQYRXRZRBR (SEQ ID NO: 8-ZRBR), RBRZRXRRXR (RBRZ-SEQ ID NO: 1), RBRRXRZRXR (SEQ ID NO: 4-ZRXR), RBRZRXRZRXR, RBRZRXRILFQYRXR(RBRZ-SEQ ID NO: 5), RBRILFQYRXRZRXR (SEQ ID NO: 8-ZRXR), RBRZRBRRXR(RBRZ-SEQ ID NO: 4), RBRRBRZRXR (SEQ ID NO: 2-ZRXR), RBRZRBRZRXR, RBRZRBRILFQYRXR(RBRZ-SEQ ID NO: 8), and RBRILFQYRBRZRXR (SEQ ID NO: 6-ZRXR), or a functional derivative thereof.

8. A construct according to claim 1 wherein the targeting-delivery peptide is selected from MSP, HSP, AAV6, AAV8 and TAT or a functional derivative thereof.

9. A construct according to claim 8 wherein the MSP peptide is ASSLNIA (SEQ ID NO: 45) or a functional derivative thereof, the HSP peptide is SKTFNTHPQSTP (SEQ ID NO: 46) or a functional derivative thereof, the AAV6 peptide is TVAVNLQSSSTDPATGDVHVM (SEQ ID NO: 47) or a functional derivative thereof, the AAV8 peptide is IVADNLQQQNTAPQIGTVNSQ (SEQ ID NO: 48) or a functional derivative thereof or the TAT peptide is YGRKKRRQRRRP (SEQ ID NO: 49) or a functional derivative thereof.

10. A construct according to claim 5 wherein the functional derivative is a polypeptide with a sequence which has homology to any of the specific sequences mentioned in claims 5 to 9 and which is able to improve delivery of the compound into cells.

11. A construct according to claim 1 wherein the construct comprises the sequences shown in any of SEQ ID NOs: 50 to 54.

12. A construct according to claim 1 wherein the cells are cardiac muscle, skeletal muscle, smooth muscle or contractile cells.

13. A construct according to claim 1 wherein the biologically active compound comprises a nucleic acid, a DNA molecule, a peptide, a protein, a DNAzyme, a Ribozyme, a chromophore, a fluorophore, and/or a pharmaceutical.

14. A construct according to claim 13 wherein the nucleic acid comprises nucleic acid with phosphodiester, 2′O-methyl, 2′ methoxy-ethyl, phosphoramidate, methylphosphonate, and/or phosphorothioate backbone chemistry, peptide nucleic acid (PNA), phosphorodiamidate morpholino oligonucleotide (PMO), locked nucleic acid (LNA), glycol nucleic acid (GNA) and threose nucleic acid (TNA), plasmid DNA or small interfering RNA (siRNA).

15. A construct according to claim 13 wherein the nucleic acid comprises a sequence capable of targeting a sequence responsible for exon skipping in a mutated pre-mRNA at an exon to be skipped or included, wherein inducing exon skipping or inclusion corrects the expression of said mutated pre-mRNA and wherein without correction the mutated pre-mRNA fails to express functional protein.

16. A composition comprising the construct of claim 1 and a pharmaceutically acceptable carrier.

17-21. (canceled)

22. A method of delivering a biologically active compound into a cell comprising contacting said cell with a construct according to claim 1 comprising the biologically active compound.

23. (canceled)

24. A method according to claim 22, wherein the method is for treating or diagnosing a cardiac or skeletal muscle disease in a subject.

25. A method according to claim 24 wherein the skeletal muscle disease is a muscular dystrophy phenotype, optionally Duchenne muscular dystrophy (DMD).

26. A method according to claim 22 wherein the construct is administered by injection, optionally by intramuscular or intravenous injection.


SEQUENCE LISTING (177 sequences)

SEQ ID NO: 1 - protein, 6 aa, Artificial sequence
  Sequence for inclusion in positively charged peptide
RXRRXR

SEQ ID NO: 2 - protein, 6 aa, Artificial sequence
  Sequence for inclusion in positively charged peptide
RXRRXR

SEQ ID NO: 3 - protein, 6 aa, Artificial sequence
  Sequence for inclusion in positively charged peptide
RXRRXR

SEQ ID NO: 4 - protein, 6 aa, Artificial sequence
  Sequence for inclusion in positively charged peptide
RXRRXR

SEQ ID NO: 5 - protein, 11 aa, Artificial sequence
  Sequence for inclusion in positively charged peptide
RXRILFQYRXR

SEQ ID NO: 6 - protein, 11 aa, Artificial sequence
  Sequence for inclusion in positively charged peptide
RXRILFQYRXR

SEQ ID NO: 7 - protein, 11 aa, Artificial sequence
  Sequence for inclusion in positively charged peptide
RXRILFQYRXR

SEQ ID NO: 8 - protein, 11 aa, Artificial sequence
  Sequence for inclusion in positively charged peptide
RXRILFQYRXR

SEQ ID NO: 9 - protein, 9 aa, Artificial sequence
  Sequence for inclusion in positively charged peptide
RXRRXRRXR

SEQ ID NO: 10 - protein, 9 aa, Artificial sequence
  Sequence for inclusion in positively charged peptide
RXRRXRRXR

SEQ ID NO: 11 - protein, 9 aa, Artificial sequence
  Sequence for inclusion in positively charged peptide
RXRRXRRXR

SEQ ID NO: 12 - protein, 9 aa, Artificial sequence
  Sequence for inclusion in positively charged peptide
RXRRXRRXR

SEQ ID NO: 13 - protein, 9 aa, Artificial sequence
  Sequence for inclusion in positively charged peptide
RXRRXRRXR

SEQ ID NO: 14 - protein, 9 aa, Artificial sequence
  Sequence for inclusion in positively charged peptide
RXRRXRRXR

SEQ ID NO: 15 - protein, 9 aa, Artificial sequence
  Sequence for inclusion in positively charged peptide
RXRRXRRXR

SEQ ID NO: 16 - protein, 9 aa, Artificial sequence
  Sequence for inclusion in positively charged peptide
RXRRXRRXR

SEQ ID NO: 17 - protein, 14 aa, Artificial sequence
  Sequence for inclusion in positively charged peptide
RXRILFQYRXRRXR

SEQ ID NO: 18 - protein, 14 aa, Artificial sequence
  Sequence for inclusion in positively charged peptide
RXRRXRILFQYRXR

SEQ ID NO: 19 - protein, 19 aa, Artificial sequence
  Sequence for inclusion in positively charged peptide
RXRILFQYRXRILFQYRXR

SEQ ID NO: 20 - protein, 14 aa, Artificial sequence
  Sequence for inclusion in positively charged peptide
RXRILFQYRXRRXR

SEQ ID NO: 21 - protein, 14 aa, Artificial sequence
  Sequence for inclusion in positively charged peptide
RXRRXRILFQYRXR

SEQ ID NO: 22 - protein, 7 aa, Artificial sequence
  Sequence for inclusion in positively charged peptide
RXRYRXR

SEQ ID NO: 23 - protein, 19 aa, Artificial sequence
  Sequence for inclusion in positively charged peptide
RXRILFQYRXRILFQYRXR

SEQ ID NO: 24 - protein, 14 aa, Artificial sequence
  Sequence for inclusion in positively charged peptide
RXRILFQYRXRRXR

SEQ ID NO: 25 - protein, 14 aa, Artificial sequence
  Sequence for inclusion in positively charged peptide
RXRRXRILFQYRXR

SEQ ID NO: 26 - protein, 19 aa, Artificial sequence
  Sequence for inclusion in positively charged peptide
RXRILFQYRXRILFQYRXR

SEQ ID NO: 27 - protein, 14 aa, Artificial sequence
  Sequence for inclusion in positively charged peptide
RXRILFQYRXRRXR

SEQ ID NO: 28 - protein, 14 aa, Artificial sequence
  Sequence for inclusion in positively charged peptide
RXRRXRILFQYRXR

SEQ ID NO: 29 - protein, 19 aa, Artificial sequence
  Sequence for inclusion in positively charged peptide
RXRILFQYRXRILFQYRXR

SEQ ID NO: 30 - protein, 14 aa, Artificial sequence
  Sequence for inclusion in positively charged peptide
RXRILFQYRXRRXR

SEQ ID NO: 31 - protein, 14 aa, Artificial sequence
  Sequence for inclusion in positively charged peptide
RXRRXRILFQYRXR

SEQ ID NO: 32 - protein, 19 aa, Artificial sequence
  Sequence for inclusion in positively charged peptide
RXRILFQYRXRILFQYRXR

SEQ ID NO: 33 - protein, 14 aa, Artificial sequence
  Sequence for inclusion in positively charged peptide
RXRILFQYRXRRXR

SEQ ID NO: 34 - protein, 14 aa, Artificial sequence
  Sequence for inclusion in positively charged peptide
RXRRXRILFQYRXR

SEQ ID NO: 35 - protein, 19 aa, Artificial sequence
  Sequence for inclusion in positively charged peptide
RXRILFQYRXRILFQYRXR

SEQ ID NO: 36 - protein, 14 aa, Artificial sequence
  Sequence for inclusion in positively charged peptide
RXRILFQYRXRRXR

SEQ ID NO: 37 - protein, 14 aa, Artificial sequence
  Sequence for inclusion in positively charged peptide
RXRRXRILFQYRXR

SEQ ID NO: 38 - protein, 19 aa, Artificial sequence
  Sequence for inclusion in positively charged peptide
RXRILFQYRXRILFQYRXR

SEQ ID NO: 39 - protein, 14 aa, Artificial sequence
  Sequence for inclusion in positively charged peptide
RXRILFQYRXRRXR

SEQ ID NO: 40 - protein, 14 aa, Artificial sequence
  Sequence for inclusion in positively charged peptide
RXRRXRILFQYRXR

SEQ ID NO: 41 - protein, 19 aa, Artificial sequence
  Sequence for inclusion in positively charged peptide
RXRILFQYRXRILFQYRXR

SEQ ID NO: 42 - protein, 12 aa, Artificial sequence
  Sequence for inclusion in positively charged peptide
RXRRXRRXRRXR

SEQ ID NO: 43 - protein, 22 aa, Artificial sequence
  Sequence for inclusion in positively charged peptide
RXRRXRRXRILFQYRXRXRXRX

SEQ ID NO: 44 - protein, 14 aa, Artificial sequence
  Sequence for inclusion in positively charged peptide
RXRRXRRXRRXRXX

SEQ ID NO: 45 - protein, 7 aa, Artificial sequence
  Sequence for inclusion in target-delivery peptide
ASSLNIA

SEQ ID NO: 46 - protein, 12 aa, Artificial sequence
  Sequence for inclusion in target-delivery peptide
SKTFNTHPQSTP

SEQ ID NO: 47 - protein, 21 aa, Artificial sequence
  Sequence for inclusion in target-delivery peptide
TVAVNLQSSSTDPATGDVHVM

SEQ ID NO: 48 - protein, 21 aa, Artificial sequence
  Sequence for inclusion in target-delivery peptide
IVADNLQQQNTAPQIGTVNSQ

SEQ ID NO: 49 - protein, 12 aa, Artificial sequence
  Sequence for inclusion in target-delivery peptide
YGRKKRRQRRRP

SEQ ID NO: 50 - protein, 23 aa, Artificial sequence
  Chimeric sequence
RXRRXRRXRRXRXXASSLNIAXC

SEQ ID NO: 51 - protein, 31 aa, Artificial sequence
  Chimeric sequence
RXRRXRRXRILFQYRXRXRXRXASSLNIAXC

SEQ ID NO: 52 - protein, 25 aa, Artificial sequence
  Chimeric sequence
RXRRXRRXRASSLNIARXRXRXRXC

SEQ ID NO: 53 - protein, 22 aa, Artificial sequence
  Chimeric sequence
RXRRXRRXRRXRXXASSLNIAX

SEQ ID NO: 54 - DNA, 25 bp, Artificial sequence
  PMO sequence used in Examples
ggccaaacctcggcttacctgaaat

SEQ ID NO: 55 - RNA, 30 nt, Artificial sequence
  Exon/intron boundary sequence that can be targeted
ccauuuugugaauguuuucuuuugaacauc

SEQ ID NO: 56 - RNA, 21 nt, Artificial sequence
  Exon/intron boundary sequence that can be targeted
guaggucacugaagagguucu

SEQ ID NO: 57 - RNA, 30 nt, Artificial sequence
  Exon/intron boundary sequence that can be targeted
uguucagggcaugaacucuuguggauccuu

SEQ ID NO: 58 - RNA, 31 nt, Artificial sequence
  Exon/intron boundary sequence that can be targeted
ucaguuuaugauuuccaucuacgaugucagu

SEQ ID NO: 59 - RNA, 23 nt, Artificial sequence
  Exon/intron boundary sequence that can be targeted
uacgaguugauugucggacccag

SEQ ID NO: 60 - RNA, 23 nt, Artificial sequence
  Exon/intron boundary sequence that can be targeted
ugcauguuccagucguugugugg

SEQ ID NO: 61 - RNA, 29 nt, Artificial sequence
  Exon/intron boundary sequence that can be targeted
cccugugcuagacugaccgugaucugcag

SEQ ID NO: 62 - RNA, 24 nt, Artificial sequence
  Exon/intron boundary sequence that can be targeted
ucuucuguuuuuguuagccaguca

SEQ ID NO: 63 - RNA, 24 nt, Artificial sequence
  Exon/intron boundary sequence that can be targeted
cagcaguugcgugaucuccacuag

SEQ ID NO: 64 - RNA, 30 nt, Artificial sequence
  Exon/intron boundary sequence that can be targeted
guaaaagaacccagcggucuucuguccauc

SEQ ID NO: 65 - RNA, 24 nt, Artificial sequence
  Exon/intron boundary sequence that can be targeted
ucuuuaaagccaguugugugaauc

SEQ ID NO: 66 - RNA, 31 nt, Artificial sequence
  Exon/intron boundary sequence that can be targeted
cuagauccgcuuuuaaaaccuguuaaaacaa

SEQ ID NO: 67 - RNA, 30 nt, Artificial sequence
  Exon/intron boundary sequence that can be targeted
cagcuucugagcgaguaauccagcugugaa

SEQ ID NO: 68 - RNA, 31 nt, Artificial sequence
  Exon/intron boundary sequence that can be targeted
gccugagcugaucugcuggcaucuugcaguu

SEQ ID NO: 69 - RNA, 22 nt, Artificial sequence
  Exon/intron boundary sequence that can be targeted
cugcaauuccccgagucucugc

SEQ ID NO: 70 - RNA, 30 nt, Artificial sequence
  Exon/intron boundary sequence that can be targeted
cggcuaauuucagagggcgcuuucuucgac

SEQ ID NO: 71 - RNA, 23 nt, Artificial sequence
  Exon/intron boundary sequence that can be targeted
caagggcaggccauuccuccuuc

SEQ ID NO: 72 - RNA, 25 nt, Artificial sequence
  Exon/intron boundary sequence that can be targeted
uugaguucugucucaagucucgaag

SEQ ID NO: 73 - RNA, 25 nt, Artificial sequence
  Exon/intron boundary sequence that can be targeted
uuaaggccucuugugcuacaggugg

SEQ ID NO: 74 - RNA, 26 nt, Artificial sequence
  Exon/intron boundary sequence that can be targeted
cagagauuuccucagcuccgccagga

SEQ ID NO: 75 - RNA, 25 nt, Artificial sequence
  Exon/intron boundary sequence that can be targeted
uccgccaucuguuagggucugugcc

SEQ ID NO: 76 - RNA, 26 nt, Artificial sequence
  Exon/intron boundary sequence that can be targeted
uccugggcagacuggaugcucuguuc

SEQ ID NO: 77 - RNA, 25 nt, Artificial sequence
  Exon/intron boundary sequence that can be targeted
uaguuucugaaauaacauauaccug

SEQ ID NO: 78 - RNA, 30 nt, Artificial sequence
  Exon/intron boundary sequence that can be targeted
cuuguagacgcugcucaaaauuggcugguu

SEQ ID NO: 79 - RNA, 25 nt, Artificial sequence
  Exon/intron boundary sequence that can be targeted
ccgucugcuuuuucuguacaaucug

SEQ ID NO: 80 - RNA, 30 nt, Artificial sequence
  Exon/intron boundary sequence that can be targeted
ucugugauacucuucaggugcaccuucugu

SEQ ID NO: 81 - RNA, 24 nt, Artificial sequence
  Exon/intron boundary sequence that can be targeted
uucugugugaaauggcugcaaauc

SEQ ID NO: 82 - RNA, 25 nt, Artificial sequence
  Exon/intron boundary sequence that can be targeted
ugaagucuuccucuuucagauucac

SEQ ID NO: 83 - RNA, 30 nt, Artificial sequence
  Exon/intron boundary sequence that can be targeted
uuuccucucgcuuucucucaucugugauuc

SEQ ID NO: 84 - RNA, 26 nt, Artificial sequence
  Exon/intron boundary sequence that can be targeted
caagcccucagcuugccuacgcacug

SEQ ID NO: 85 - RNA, 27 nt, Artificial sequence
  Exon/intron boundary sequence that can be targeted
aucguuucuucacggacagugugcugg

SEQ ID NO: 86 - RNA, 30 nt, Artificial sequence
  Exon/intron boundary sequence that can be targeted
caggggcaacucuuccaccaguaacugaaa

SEQ ID NO: 87 - RNA, 27 nt, Artificial sequence
  Exon/intron boundary sequence that can be targeted
cugcuauuucaguuuccuggggaaaag

SEQ ID NO: 88 - RNA, 25 nt, Artificial sequence
  Exon/intron boundary sequence that can be targeted
acaucaaggaagauggcauuucuag

SEQ ID NO: 89 - RNA, 30 nt, Artificial sequence
  Exon/intron boundary sequence that can be targeted
uccaacuggggacgccucuguuccaaaucc

SEQ ID NO: 90 - RNA, 31 nt, Artificial sequence
  Exon/intron boundary sequence that can be targeted
cauucaacuguugccuccgguucugaaggug

SEQ ID NO: 91 - RNA, 27 nt, Artificial sequence
  Exon/intron boundary sequence that can be targeted
gugugaaagcugaggggacgaggcagg

SEQ ID NO: 92 - RNA, 25 nt, Artificial sequence
  Exon/intron boundary sequence that can be targeted
cgaggcuggcucaggggggaguccu

SEQ ID NO: 93 - RNA, 25 nt, Artificial sequence
  Exon/intron boundary sequence that can be targeted
ggacaggccuuuauguucgugcugc

SEQ ID NO: 94 - RNA, 27 nt, Artificial sequence
  Exon/intron boundary sequence that can be targeted
cugugcuuguguccuggggaggacuga

SEQ ID NO: 95 - RNA, 26 nt, Artificial sequence
  Exon/intron boundary sequence that can be targeted
ucucauuggcuuuccagggguauuuc

SEQ ID NO: 96 - RNA, 23 nt, Artificial sequence
  Exon/intron boundary sequence that can be targeted
caucuucugauaauuuuccuguu

SEQ ID NO: 97 - RNA, 23 nt, Artificial sequence
  Exon/intron boundary sequence that can be targeted
gucugcauccaggaacauggguc

SEQ ID NO: 98 - RNA, 30 nt, Artificial sequence
  Exon/intron boundary sequence that can be targeted
ugugaugugguccacauucuggucaaaagu

SEQ ID NO: 99 - RNA, 22 nt, Artificial sequence
  Exon/intron boundary sequence that can be targeted
cuuugagaccucaaauccuguu

SEQ ID NO: 100 - RNA, 20 nt, Artificial sequence
  Exon/intron boundary sequence that can be targeted
ggagagagcuuccuguagcu

SEQ ID NO: 101 - RNA, 24 nt, Artificial sequence
  Exon/intron boundary sequence that can be targeted
uguucagcuucuguuagccacuga

SEQ ID NO: 102 - RNA, 31 nt, Artificial sequence
  Exon/intron boundary sequence that can be targeted
caagcuuuucuuuuaguugcugcucuuuucc

SEQ ID NO: 103 - RNA, 30 nt, Artificial sequence
  Exon/intron boundary sequence that can be targeted
uucucagguaaagcucuggaaaccugaaag

SEQ ID NO: 104 - RNA, 30 nt, Artificial sequence
  Exon/intron boundary sequence that can be targeted
cuggcuuccaaaugggaccugaaaaagaac

SEQ ID NO: 105 - RNA, 30 nt, Artificial sequence
  Exon/intron boundary sequence that can be targeted
cuggcgagcaagguccuugacguggcucac

SEQ ID NO: 106 - RNA, 31 nt, Artificial sequence
  Exon/intron boundary sequence that can be targeted
gggcuucaugcagcugccugacucgguccuc

SEQ ID NO: 107 - RNA, 27 nt, Artificial sequence
  Exon/intron boundary sequence that can be targeted
cauccagucuaggaagagggccgcuuc

SEQ ID NO: 108 - RNA, 24 nt, Artificial sequence
  Exon/intron boundary sequence that can be targeted
ccucuaagacagucugcacuggca

SEQ ID NO: 109 - RNA, 24 nt, Artificial sequence
  Exon/intron boundary sequence that can be targeted
aaguugaucagaguaacgggacug

SEQ ID NO: 110 - RNA, 25 nt, Artificial sequence
  Exon/intron boundary sequence that can be targeted
gauccauugcuguuuuccauuucug

SEQ ID NO: 111 - RNA, 26 nt, Artificial sequence
  Exon/intron boundary sequence that can be targeted
ccuccuuucuggcauagaccuuccac

SEQ ID NO: 112 - RNA, 26 nt, Artificial sequence
  Exon/intron boundary sequence that can be targeted
ccaaugccauccuggaguuccuguaa

SEQ ID NO: 113 - RNA, 29 nt, Artificial sequence
  Exon/intron boundary sequence that can be targeted
ccacucagagcucagaucuucuaacuucc

SEQ ID NO: 114 - RNA, 20 nt, Artificial sequence
  Exon/intron boundary sequence that can be targeted
cuuggagucuucuaggagcc

SEQ ID NO: 115 - RNA, 25 nt, Artificial sequence
  Exon/intron boundary sequence that can be targeted
guuauccaaacgucuuuguaacagg

SEQ ID NO: 116 - RNA, 25 nt, Artificial sequence
  Exon/intron boundary sequence that can be targeted
acucaugauuacacguucuuuaguu

SEQ ID NO: 117 - RNA, 22 nt, Artificial sequence
  Exon/intron boundary sequence that can be targeted
uccucaggaggcagcucuaaau

SEQ ID NO: 118 - RNA, 27 nt, Artificial sequence
  Exon/intron boundary sequence that can be targeted
gagauggcucucucccagggacccugg

SEQ ID NO: 119 - RNA, 25 nt, Artificial sequence
  Exon/intron boundary sequence that can be targeted
ugggauggucccagcaaguuguuug

SEQ ID NO: 120 - RNA, 27 nt, Artificial sequence
  Exon/intron boundary sequence that can be targeted
gcaaagggccuucugcagucuucggag

SEQ ID NO: 121 - RNA, 27 nt, Artificial sequence
  Exon/intron boundary sequence that can be targeted
gauccucccuguucguccccuauuaug

SEQ ID NO: 122 - RNA, 26 nt, Artificial sequence
  Exon/intron boundary sequence that can be targeted
gcgcuggucacaaaauccuguugaac

SEQ ID NO: 123 - RNA, 24 nt, Artificial sequence
  Exon/intron boundary sequence that can be targeted
ugcuuuagacuccuguaccugaua

SEQ ID NO: 124 - RNA, 27 nt, Artificial sequence
  Exon/intron boundary sequence that can be targeted
gcugacugcugucggaccucuguagag

SEQ ID NO: 125 - RNA, 24 nt, Artificial sequence
  Exon/intron boundary sequence that can be targeted
gauaggugguaucaacaucuguaa

SEQ ID NO: 126 - RNA, 21 nt, Artificial sequence
  Exon/intron boundary sequence that can be targeted
caggagcuuccaaaugcugca

SEQ ID NO: 127 - RNA, 22 nt, Artificial sequence
  Exon/intron boundary sequence that can be targeted
uccucagcagaaagaagccacg

SEQ ID NO: 128 - RNA, 23 nt, Artificial sequence
  Exon/intron boundary sequence that can be targeted
ugacagccugugaaaucugugag

SEQ ID NO: 129 - RNA, 28 nt, Artificial sequence
  Exon/intron boundary sequence that can be targeted
cuggcagaauucgauccaccggcuguuc

SEQ ID NO: 130 - RNA, 22 nt, Artificial sequence
  Exon/intron boundary sequence that can be targeted
cagcaguaguugucaucugcuc

SEQ ID NO: 131 - RNA, 25 nt, Artificial sequence
  Exon/intron boundary sequence that can be targeted
cauucauuuccuuucgcaucuuacg

SEQ ID NO: 132 - RNA, 26 nt, Artificial sequence
  Exon/intron boundary sequence that can be targeted
aucucuuugucaauuccauaucugua

SEQ ID NO: 133 - RNA, 23 nt, Artificial sequence
  Exon/intron boundary sequence that can be targeted
ucugcagaauaaucccggagaag

SEQ ID NO: 134 - RNA, 25 nt, Artificial sequence
  Exon/intron boundary sequence that can be targeted
gcucaagagauccacugcaaaaaac

SEQ ID NO: 135 - RNA, 25 nt, Artificial sequence
  Exon/intron boundary sequence that can be targeted
ucugcaggauauccaugggcugguc

SEQ ID NO: 136 - RNA, 26 nt, Artificial sequence
  Exon/intron boundary sequence that can be targeted
gccauacguacguaucauaaacauuc

SEQ ID NO: 137 - RNA, 25 nt, Artificial sequence
  Exon/intron boundary sequence that can be targeted
uuuaaaaccuguuaaaacaagaaag

SEQ ID NO: 138 - RNA, 31 nt, Artificial sequence; identical to SEQ ID NO: 66
  Exon/intron boundary sequence that can be targeted

SEQ ID NO: 139 - RNA, 25 nt, Artificial sequence
  Exon/intron boundary sequence that can be targeted
cuagauccgcuuuuaaaaccuguua

SEQ ID NO: 140 - RNA, 31 nt, Artificial sequence
  Exon/intron boundary sequence that can be targeted
ucuuuucuagauccgcuuuuaaaaccuguua

SEQ ID NO: 141 - RNA, 20 nt, Artificial sequence
  Exon/intron boundary sequence that can be targeted
ccgcuuuuaaaaccuguuaa

SEQ ID NO: 142 - RNA, 25 nt, Artificial sequence
  Exon/intron boundary sequence that can be targeted
ucuuuucuagauccgcuuuuaaaac

SEQ ID NO: 143 - RNA, 25 nt, Artificial sequence
  Exon/intron boundary sequence that can be targeted
cuuuuucuuuucuagauccgcuuuu

SEQ ID NO: 144 - RNA, 25 nt, Artificial sequence
  Exon/intron boundary sequence that can be targeted
gauugcuuuuucuuuucuagauccg

SEQ ID NO: 145 - RNA, 26 nt, Artificial sequence
  Exon/intron boundary sequence that can be targeted
uggauugcuuuuucuuuucuagaucc

SEQ ID NO: 146 - RNA, 23 nt, Artificial sequence
  Exon/intron boundary sequence that can be targeted
gaucuuguuugagugaauacagu

SEQ ID NO: 147 - RNA, 23 nt, Artificial sequence
  Exon/intron boundary sequence that can be targeted
ccgucuucugggucacugacuua

SEQ ID NO: 148 - RNA, 25 nt, Artificial sequence
  Exon/intron boundary sequence that can be targeted
caugcuuccgucuucugggucacug

SEQ ID NO: 149 - RNA, 22 nt, Artificial sequence
  Exon/intron boundary sequence that can be targeted
guuauccagccaugcuuccguc

SEQ ID NO: 150 - RNA, 22 nt, Artificial sequence
  Exon/intron boundary sequence that can be targeted
guaucacuaaccugugcuguac

SEQ ID NO: 151 - RNA, 25 nt, Artificial sequence
  Exon/intron boundary sequence that can be targeted
ugauaauugguaucacuaaccugug

SEQ ID NO: 152 - RNA, 31 nt, Artificial sequence; identical to SEQ ID NO: 102
  Exon/intron boundary sequence that can be targeted

SEQ ID NO: 153 - RNA, 26 nt, Artificial sequence
  Exon/intron boundary sequence that can be targeted
accagaguaacagucugaguaggagc

SEQ ID NO: 154 - RNA, 30 nt, Artificial sequence
  Exon/intron boundary sequence that can be targeted
acaucaaggaagauggcauuucuaguuugg

SEQ ID NO: 155 - RNA, 25 nt, Artificial sequence
  Exon/intron boundary sequence that can be targeted
acaucaaggaagauggcauuucuag

SEQ ID NO: 156 - RNA, 30 nt, Artificial sequence
  Exon/intron boundary sequence that can be targeted
cuccaacaucaaggaagauggcauuucuag

SEQ ID NO: 157 - RNA, 24 nt, Artificial sequence
  Exon/intron boundary sequence that can be targeted
uucuguccaagcccgguugaaauc

SEQ ID NO: 158 - RNA, 21 nt, Artificial sequence
  Exon/intron boundary sequence that can be targeted
cacccaccaucacccucugug

SEQ ID NO: 159 - RNA, 22 nt, Artificial sequence
  Exon/intron boundary sequence that can be targeted
aucaucucguugauauccucaa

SEQ ID NO: 160 - RNA, 25 nt, Artificial sequence
  Exon/intron boundary sequence that can be targeted
aucauuuuuucucauaccuucugcu

SEQ ID NO: 161 - RNA, 23 nt, Artificial sequence
  Exon/intron boundary sequence that can be targeted
cucauaccuucugcuugaugauc

SEQ ID NO: 162 - RNA, 25 nt, Artificial sequence
  Exon/intron boundary sequence that can be targeted
gauucugaauucuuucaacuagaau

SEQ ID NO: 163 - RNA, 22 nt, Artificial sequence
  Exon/intron boundary sequence that can be targeted
auucuuucaacuagaauaaaag

SEQ ID NO: 164 - RNA, 15 nt, Artificial sequence
  Exon/intron boundary sequence that can be targeted
cgaagggcgaccacc

SEQ ID NO: 165 - RNA, 24 nt, Artificial sequence
  Exon/intron boundary sequence that can be targeted
cuguugccuccgguucugaaggug

SEQ ID NO: 166 - RNA, 31 nt, Artificial sequence; identical to SEQ ID NO: 90
  Exon/intron boundary sequence that can be targeted

SEQ ID NO: 167 - RNA, 25 nt, Artificial sequence
  Exon/intron boundary sequence that can be targeted
cauucaacuguugccuccgguucug

SEQ ID NO: 168 - RNA, 22 nt, Artificial sequence
  Exon/intron boundary sequence that can be targeted
uuggcucuggccuguccuaaga

SEQ ID NO: 169 - RNA, 25 nt, Artificial sequence
  Exon/intron boundary sequence that can be targeted
guauagggacccuccuuccaugacu

SEQ ID NO: 170 - RNA, 27 nt, Artificial sequence
  Exon/intron boundary sequence that can be targeted
gguaucuuugauacuaaccuugguuuc

SEQ ID NO: 171 - RNA, 21 nt, Artificial sequence
  Exon/intron boundary sequence that can be targeted
uacuaaccuugguuucuguga

SEQ ID NO: 172 - RNA, 25 nt, Artificial sequence
  Exon/intron boundary sequence that can be targeted
ggccaaaccucggcuuaccugaaau

SEQ ID NO: 173 - RNA, 20 nt, Artificial sequence
  Exon/intron boundary sequence that can be targeted
ggccaaaccucggcuuaccu

SEQ ID NO: 174 - RNA, 30 nt, Artificial sequence
  Exon/intron boundary sequence that can be targeted
ggccaaaccucggcuuaccugaaauuuucg

SEQ ID NO: 175 - RNA, 30 nt, Artificial sequence
  Exon/intron boundary sequence that can be targeted
uuaaaggccaaaccucggcuuaccugaaau

SEQ ID NO: 176 - RNA, 31 nt, Artificial sequence
  Exon/intron boundary sequence that can be targeted
gugcuucucuguugcagaccgugccugggca

SEQ ID NO: 177 - RNA, 30 nt, Artificial sequence
  Exon/intron boundary sequence that can be targeted
gccccgauggaggcaaguuucacuuccucc